(19) United States
(12) Patent Application Publication
(10) Pub. No.: US 2024/0006052 A1
(43) Pub. Date: Jan. 4, 2024

(54) SYSTEM IMPLEMENTING GENERATIVE ADVERSARIAL NETWORK ADAPTED TO PREDICTION IN BEHAVIORAL AND/OR PHYSIOLOGICAL CONTEXTS

(71) Applicant: Cornell University, Ithaca, NY (US)

(72) Inventors: Daniel A. Adler, Yardley, PA (US); Tanzeem Choudhury, Pittsford, NY (US); Vincent W.-S. Tseng, Boston, MA (US); Gengmo Qi, Ithaca, NY (US)

(21) Appl. No.: 18/026,371

(22) PCT Filed: Sep. 27, 2021
(86) PCT No.: PCT/US2021/052203

§ 371 Date: Mar. 15, 2023

Related U.S. Application Data
(60) Provisional application No. 63/083,234, filed on Sep. 25, 2020.

(51) Int. Cl. G16H 20/70; G16H 50/20; G16H 40/63

(52) U.S. Cl. CPC G16H 20/70; G16H 40/63; G16H 50/20

(57) ABSTRACT

A method comprises obtaining data characterizing a given subject over time, applying at least a portion of the obtained data to a generative adversarial network adapted to generate a prediction of at least one change in at least one of behavior and physiology of the given subject from the obtained data, and executing at least one automated remedial action relating to the given subject based at least in part on the generated prediction. The generative adversarial network is configured to implement multi-task learning, across a plurality of subjects, in which changes in multiple distinct features are treated as separate but linked tasks. The generative adversarial network comprises separate discriminators for each of the multiple distinct features and separate discriminators for each of a plurality of different clusters of respective subsets of the plurality of subjects, and combines outputs of respective ones of the discriminators for the features and the clusters in generating the prediction.

### RELATED APPLICATION

The present application claims priority to U.S. Provisional patent application Ser. No. 63/083,234, filed Sep. 25, 2020, which is incorporated by reference herein in its entirety.

### FIELD

The field relates generally to information processing systems, and more particularly to machine learning and other types of artificial intelligence implemented in such systems.

### BACKGROUND

Behavioral and/or physiological analysis is fundamental in numerous information processing contexts, including diverse fields such as healthcare, security and sports. Conventional approaches to behavioral and/or physiological analysis are problematic in that such approaches often require extensive manual intervention by highly trained personnel, and can therefore lead to excessive costs and other difficulties in analyzing both simple and complex behaviors and/or physiologies in a repeatable and scalable manner.

### SUMMARY

Illustrative embodiments provide systems implementing generative adversarial networks (GANs) adapted to prediction in behavioral contexts, physiological contexts, and/or in numerous other contexts. For example, some embodiments provide a system adapting one or more GANs to predict behavior, physiology, and well-being changes associated with a specific life event, illustratively using passive and active sensing data. One or more such embodiments illustratively further provide various types of automated remediation responsive to predictions generated by the one or more GANs. For example, some embodiments implement GAN-based prediction and remediation algorithms to at least partially automate various aspects of patient care in healthcare applications such as telemedicine. Such applications can involve a wide variety of different types of remote medical monitoring and intervention.

In one embodiment, a method comprises obtaining data characterizing a given subject over time, applying at least a portion of the obtained data to a GAN adapted to generate a prediction of at least one change in at least one of behavior and physiology of the given subject from the obtained data, and executing at least one automated remedial action relating to the given subject based at least in part on the generated prediction. The GAN is configured to implement multi-task learning (MTL), across a plurality of subjects, in which changes in multiple distinct features are treated as separate but linked tasks. The GAN comprises separate discriminators for each of the multiple distinct features and separate discriminators for each of a plurality of different clusters of respective subsets of the plurality of subjects, and combines outputs of respective ones of the discriminators for the features and outputs of respective ones of the discriminators for the clusters in generating the prediction for the given subject.

In some embodiments, the multiple distinct features comprise, for example, one or more of a heart rate measure, a mood measure, a sleep measure and an activity measure, and the generated prediction comprises an indicator of resilience of the given subject under stress, or under one or more other specified conditions. The generated prediction is illustratively associated with one or more predicted changes in mental health of the given subject, so as to permit interpretation of the generated prediction in the context of the mental health of the given subject. Numerous other arrangements of features and generated predictions are possible. For example, in some embodiments, features can comprise respective multiple distinct data types, and the term “feature” as used herein is therefore intended to be broadly construed.

In some embodiments, executing at least one automated remedial action relating to the subject based at least in part on the generated prediction illustratively comprises generating at least one output signal in a telemedicine application. For example, such output signals in a telemedicine application can comprise a prediction visualization signal for presentation on a user terminal, diagnosis information transmitted over a network to a medical professional, and/or prescription information transmitted over a network to a prescription-filling entity. A wide variety of other signals can be generated in conjunction with execution of one or more automated remedial actions in illustrative embodiments. For example, one or more prediction-driven control signals can be integrated into behavioral and/or wearable technologies for self-intervention. This illustratively includes utilizing control signals generated in the manner disclosed herein to provide a user with recommendations for behavioral interventions via a smartphone, wearable or other type of user device.

It is to be appreciated that the foregoing arrangements are only examples, and numerous alternative arrangements are possible.

These and other illustrative embodiments include but are not limited to systems, methods, apparatus, processing devices, integrated circuits, and computer program products comprising processor-readable storage media having software program code embodied therein.

### BRIEF DESCRIPTION OF THE FIGURES

FIG. 1 shows an information processing system comprising a processing platform implementing a GAN adapted to prediction in contexts such as behavior and physiology in an illustrative embodiment.

FIGS. 2A and 2B show respective example training and testing processes in illustrative embodiments. These figures are collectively referred to herein as FIG. 2.

FIG. 3A shows example configurations of GANs in illustrative embodiments, and FIG. 3B shows a more detailed view of a particular one of the GANs in an illustrative embodiment. These figures are collectively referred to herein as FIG. 3.

FIG. 4 shows distributions of effect sizes for training and testing data in an illustrative embodiment.

FIG. 5 shows individual model results across participants in illustrative embodiments.

FIG. 6 shows example effect size prediction results on a test set for a particular feature.

FIG. 7 summarizes example indicators utilized in illustrative embodiments.

FIG. 8 shows depression symptom change trajectories from a 4-class model in an illustrative embodiment.

FIG. 9 shows an example analysis pipeline in an illustrative embodiment.

FIG. 10 shows plots of shared significant coefficients using calculated features from actual and predicted data in an illustrative embodiment.

### DETAILED DESCRIPTION

Illustrative embodiments can be implemented, for example, in the form of information processing systems comprising one or more processing platforms each having at least one computer, server or other processing device. A number of examples of such systems will be described in detail herein. It should be understood, however, that embodiments of the invention are more generally applicable to a wide variety of other types of information processing systems and associated computers, servers or other processing devices or other components. Accordingly, the term “information processing system” as used herein is intended to be broadly construed so as to encompass these and other arrangements.

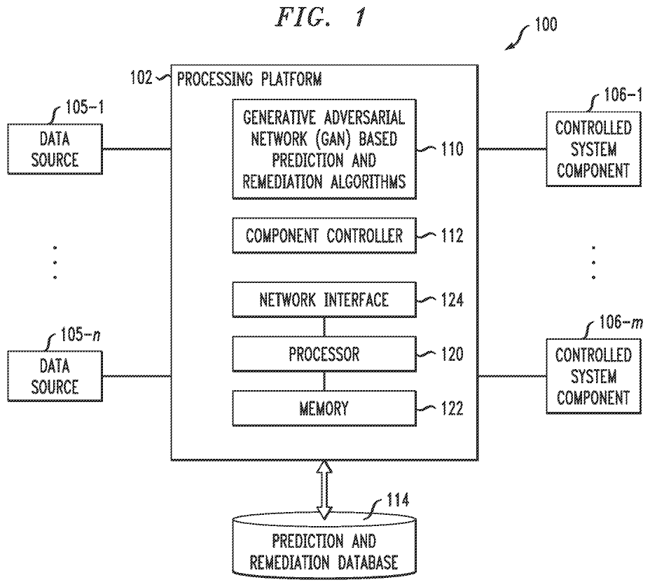

FIG. 1 shows an information processing system 100 implementing a GAN adapted to prediction in contexts such as behavior and/or physiology in an illustrative embodiment. The system 100 comprises a processing platform 102. Coupled to the processing platform 102 are data sources 105-1, . . . 105-n and controlled system components 106-1, . . . 106-m, where n and m are arbitrary integers greater than or equal to two and may but need not be equal. Other embodiments can include only a single data source and/or only a single controlled system component. The processing platform 102 implements one or more GAN-based algorithms 110 and at least one component controller 112. The GAN-based algorithms 110 in the present embodiment more particularly comprise GAN-based prediction and remediation algorithms, although other arrangements are possible.

In operation, the processing platform 102 is illustratively configured to obtain, from one or more of the data sources 105, data characterizing a given subject over time, to apply at least a portion of the obtained data to at least one GAN implemented in the GAN-based algorithms 110 to generate a prediction of at least one change in at least one of behavior and physiology of the given subject from the obtained data, and to execute at least one automated remedial action relating to the given subject based at least in part on the generated prediction, illustratively via the component controller 112.

For example, the data may be obtained from at least one of one or more wearable devices of the given subject, a smartphone of the given subject, and one or more sensors associated with the given subject. The generated prediction can comprise, for example, an indicator of resilience of the given subject, although a wide variety of other types of predictions can be generated using the GAN-based algorithms 110 in other embodiments.

A given GAN implemented in processing platform 102 is illustratively configured to implement MTL, across a plurality of subjects, in which changes in multiple distinct features are treated as separate but linked tasks. The GAN in some embodiments comprises separate discriminators for each of the multiple distinct features and separate discriminators for each of a plurality of different clusters of respective subsets of the plurality of subjects, and is further configured to combine outputs of respective ones of the discriminators for the features and outputs of respective ones of the discriminators for the clusters in generating the prediction for the given subject.

The GAN in some embodiments can further include, in addition to distinct discriminators per cluster, distinct output layers of the generator per cluster. Such arrangements facilitate MTL across a plurality of subjects.

In some embodiments, the multiple distinct features comprise, for example, one or more of a heart rate measure, a mood measure, a sleep measure and an activity measure, and the generated prediction comprises an indicator of resilience of the given subject under stress, or under one or more other specified conditions. The generated prediction is illustratively associated with one or more predicted changes in mental health of the given subject, so as to permit interpretation of the generated prediction in the context of the mental health of the given subject. Numerous other arrangements of features and generated predictions are possible. For example, in other embodiments, the changes predicted in the system 100 could involve any life event, whether stressful or non-stressful.

Also, it is to be appreciated that the term “feature” as used herein is intended to be broadly construed, and should not be viewed as being limited in any way to the particular features mentioned above or elsewhere herein. For example, in some embodiments, features can comprise respective multiple distinct data types.

The GAN in some embodiments implements an adversarial loss function that characterizes the generated prediction utilizing a clinically interpretable metric.

Additionally or alternatively, the clusters of respective subsets of the plurality of subjects are determined by applying, for example, a “k-means” clustering algorithm utilizing a clinically interpretable metric. Other types of clustering algorithms can be used in other embodiments, as will be apparent to those skilled in the art.

An example of the above-noted clinically interpretable metric is Cohen's d, although other metrics can be used. In some embodiments herein, Cohen's d is more particularly referred to as Cohen's dₛ or “effect size.”

It is to be appreciated that the term “GAN-based algorithm” as used herein is intended to be broadly construed to encompass a prediction algorithm and/or a remediation algorithm operating at least in part utilizing a GAN. Detailed examples of particular implementations of GAN-based algorithms 110 are described in detail elsewhere herein.

The component controller 112 generates one or more control signals for adjusting, triggering or otherwise controlling various operating parameters associated with the controlled system components 106 based at least in part on predictions generated by the GAN-based algorithms 110. A wide variety of different types of devices or other components can be controlled by component controller 112, possibly by applying control signals or other signals or information thereto, including additional or alternative components that are part of the same processing device or set of processing devices that implement the processing platform 102. Such control signals, and additionally or alternatively other types of signals and/or information, can be communicated over one or more networks to other processing devices, such as user terminals associated with respective system users.

The processing platform 102 is configured to utilize a prediction and remediation database 114. Such a database illustratively stores user data, user profiles and a wide variety of other types of information, including data from one or more of the data sources 105, that may be utilized by the GAN-based algorithms 110 in performing prediction and remediation operations. The prediction and remediation database 114 is also configured to store related information, including various processing results, such as predictions or other outputs generated by the GAN-based algorithms 110.

The component controller 112 utilizes outputs generated by the GAN-based algorithms 110 to control one or more of the controlled system components 106. The controlled system components 106 in some embodiments therefore comprise system components that are driven at least in part by outputs generated by the GAN-based algorithms 110. For example, a controlled component can comprise a processing device such as a computer, a mobile telephone or a wearable device that presents a display to a user and/or directs a user to adjust its behavior in a particular manner responsive to an output of a GAN-based algorithm. These and numerous other different types of controlled system components 106 can make use of outputs generated by the GAN-based algorithms 110, including various types of equipment and other systems associated with one or more of the example use cases described elsewhere herein.

Although the GAN-based algorithms 110 and the component controller 112 are both shown as being implemented on processing platform 102 in the present embodiment, this is by way of illustrative example only. In other embodiments, the GAN-based algorithms 110 and the component controller 112 can each be implemented on a separate processing platform. A given such processing platform is assumed to include at least one processing device comprising a processor coupled to a memory.

Examples of such processing devices include computers, servers or other processing devices arranged to communicate over a network. Storage devices such as storage arrays or cloud-based storage systems used for implementation of prediction and remediation database 114 are also considered “processing devices” as that term is broadly used herein.

The network can comprise, for example, a global computer network such as the Internet, a wide area network (WAN), a local area network (LAN), a satellite network, a telephone or cable network, a cellular network such as a 3G, 4G or 5G network, a wireless network implemented using a wireless protocol such as Bluetooth, WiFi or WiMAX, or various portions or combinations of these and other types of communication networks.

It is also possible that at least portions of other system elements such as one or more of the data sources 105 and/or the controlled system components 106 can be implemented as part of the processing platform 102, although shown as being separate from the processing platform 102 in the figure.

For example, in some embodiments, the system 100 can comprise a laptop computer, tablet computer or desktop personal computer, a mobile telephone, a wearable device, or another type of computer or communication device, as well as combinations of multiple such processing devices, configured to incorporate at least one data source and to execute a GAN-based algorithm for controlling at least one system component.

Examples of automated remedial actions that may be taken in the processing platform 102 responsive to outputs generated by the GAN-based algorithms 110 include generating in the component controller 112 at least one control signal for controlling at least one of the controlled system components 106 over a network, generating at least a portion of at least one output display for presentation on at least one user terminal, generating an alert for delivery to at least user terminal over a network, and/or storing the outputs in the prediction and remediation database 114.

A wide variety of additional or alternative automated remedial actions may be taken in other embodiments. The particular automated remedial action or actions will tend to vary depending upon the particular use case in which the system 100 is deployed.

For example, some embodiments implement GAN-based prediction and remediation algorithms to at least partially automate various aspects of patient care in healthcare applications such as telemedicine. Such applications illustratively involve a wide variety of different types of remote medical monitoring and intervention.

An example of an automated remedial action in this particular context includes generating at least one output signal, such as a prediction visualization signal for presentation on a user terminal, diagnosis information transmitted over a network to a medical professional, and/or prescription information transmitted over a network to a pharmacy or other prescription-filling entity.

Another example of an automated remedial action includes integrating one or more prediction-driven control signals into behavioral and/or wearable technologies for self-intervention. In a more particular example of such an arrangement, control signals generated in system 100 are utilized in a smartphone, wearable or other user device for providing a user with recommendations for behavioral interventions.

In some embodiments, the system 100 is configured to predict changes in behavioral, physiology, mood and/or other characteristics that are associated with mental health symptom development, rather than predicting symptom development itself. For example, instead of simply predicting symptoms (e.g., “you're going to develop depression”), the system 100 in illustrative embodiments is advantageously configured to direct useful interventions (e.g., “we believe your sleep is going to change, and that is associated with depression symptom development, this is how you can work on improving your sleep”).

Additional examples of such use cases are provided elsewhere herein. It is to be appreciated that the term “automated remedial action” as used herein is intended to be broadly construed, so as to encompass the above-described automated remedial actions, as well as numerous other actions that are automatically driven based at least in part on predictions generating using a GAN-based prediction algorithm as disclosed herein, with such actions being configured to address or otherwise remediate various conditions indicated by the corresponding predictions.

The processing platform 102 in the present embodiment further comprises a processor 120, a memory 122 and a network interface 124. The processor 120 is assumed to be operatively coupled to the memory 122 and to the network interface 124 as illustrated by the interconnections shown in the figure.

The processor 120 may comprise, for example, a microprocessor, an application-specific integrated circuit (ASIC), a field-programmable gate array (FPGA), a central processing unit (CPU), a graphics processing unit (GPU), a tensor processing unit (TPU), an arithmetic logic unit (ALU), a digital signal processor (DSP), or other similar processing device component, as well as other types and arrangements of processing circuitry, in any combination. At least a portion of the functionality of at least one GAN or an associated GAN-based prediction and/or remediation algorithm provided by one or more processing devices as disclosed herein can be implemented using such circuitry.

In some embodiments, the processor 120 comprises one or more graphics processor integrated circuits. Such graphics processor integrated circuits are illustratively implemented in the form of one or more GPUs. Accordingly, in some embodiments, system 100 is configured to include a GPU-based processing platform. Such a GPU-based processing platform can be cloud-based configured to implement one or more GANs for processing data associated with a large number of system users. Similar arrangements can be implemented using TPUs and/or other processing devices.

Numerous other arrangements are possible. For example, in some embodiments, a GAN and its associated GAN-based algorithm can be implemented on a single processor-based device, such as a smartphone, client computer or other user device, utilizing one or more processors of that device. Such embodiments are also referred to herein as “on-device” implementations of GAN-based algorithms.

The memory 122 stores software program code for execution by the processor 120 in implementing portions of the functionality of the processing platform 102. For example, at least portions of the functionality of GAN-based algorithms 110 and component controller 112 can be implemented using program code stored in memory 122.

A given such memory that stores such program code for execution by a corresponding processor is an example of what is more generally referred to herein as a processor-readable storage medium having program code embodied therein, and may comprise, for example, electronic memory such as SRAM, DRAM or other types of random access memory, flash memory, read-only memory (ROM), magnetic memory, optical memory, or other types of storage devices in any combination.

Articles of manufacture comprising such processor-readable storage media are considered embodiments of the invention. The term “article of manufacture” as used herein should be understood to exclude transitory, propagating signals.

Other types of computer program products comprising processor-readable storage media can be implemented in other embodiments.

In addition, illustrative embodiments may be implemented in the form of integrated circuits comprising processing circuitry configured to implement processing operations associated with one or both of the GAN-based algorithms 110 and the component controller 112 as well as other related functionality. For example, at least a portion of a GAN of system 100 is illustratively implemented in at least one neural network integrated circuit of a processing device of the processing platform 102.

The network interface 124 is configured to allow the processing platform 102 to communicate over one or more networks with other system elements, and may comprise one or more conventional transceivers.

It is to be appreciated that the particular arrangement of components and other system elements shown in FIG. 1 is presented by way of illustrative example only, and numerous alternative embodiments are possible. For example, other embodiments of information processing systems can be configured to implement GAN-based algorithm functionality of the type disclosed herein.

Terms such as “data source” and “controlled system component” as used herein are intended to be broadly construed. For example, a given set of data sources in some embodiments can comprise one or more wearable devices of a subject, a smartphone of the subject, and/or one or more sensors associated with the subject. Additionally or alternatively, data sources can comprise video cameras, sensor arrays or other types of imaging or data capture devices. Other examples of data sources include sources of data indicative of online behavior, such as data collected from social media sites and web browsers, or other sources of behavioral data. This includes various types of databases or other storage systems accessible over a network. A wide variety of different types of data sources can be used to provide input data to a GAN-based algorithm in illustrative embodiments. A given controlled component can illustratively comprise a computer, a mobile telephone, a wearable device or other type of processing device that receives an output from a GAN-based algorithm and performs at least one automated remedial action in response thereto.

Illustrative embodiments of the system 100 can be configured, for example, to predict individual-level behavior, physiology and well-being changes that occur around a life event, collected via sensors associated with wearable devices, smartphones, etc. Such embodiments can associate the predicted behavior, physiology, and well-being changes to changes in mental health symptoms. The predicted behavior, physiology, and well-being changes can thus be interpreted within the context of mental health changes.

Additionally or alternatively, some embodiments are configured to implement a GAN that uses MTL to predict fine-grained individual-level changes, and multisensor feature changes (e.g., the behavior, physiology and well-being features).

A GAN in these or other embodiments can be illustratively configured to incorporate a novel generative adversarial loss function that measures how “real” the predicted changes are, using a clinically interpretable metric, such as Cohen's d.

The system 100 can be configured to support a wide variety of distinct applications, in numerous diverse contexts.

For example, the system 100 can allow users to visualize their predicted behavior, physiology, and well-being changes around a specific, upcoming stressful life event. With automated assistance from the system 100, users can visualize how the predicted changes are associated with mental health symptom changes, and/or be provided with intervention or other treatment in an automated manner before mental health deteriorates based upon the system's specific predicted behavior, physiology, and well-being changes and the modeled associations. The system 100 can generate personalized intervention suggestions based upon the predicted changes.

For example, the following are use cases of how an individual user (a “patient”) can interact with the system 100.

Individual A is about to start a new job. The system 100 uses data collected off of Individual A's smartphone to predict that Individual A is likely going to experience a decrease in the amount of daily sleep after beginning the job, and shows that sleep decreases are linked to increased anxiety symptoms. With automated assistance from system 100, Individual A is able to find a sleep coach prior to starting a new job.

Individual B is about to have a child. The system 100 uses data collected off of Individual B's wearable device (e.g., Fitbit) to predict that Individual B is going to experience a decrease in physical activity when their child arrives, and that a decrease in physical activity is associated with an increase in depression symptoms. With automated assistance from system 100, Individual B consults with their physician, who speaks to them about creative ways to integrate physical activity into their life. Their physician also recommends beginning therapy.

Additionally or alternatively, system 100 can be configured to allow clinicians to predict their patients' behavior, physiology, and well-being changes remotely when they know their patient is about to experience novel stress.

For example, system 100 allows clinicians to use the modeled associations between behavior, physiology, and well-being changes to introduce targeted interventions to sustain mental health under stress, or under other specified conditions, thereby improving resilience.

Clinicians can develop personalized treatments based upon the specific predictions of the system, and the link between these predictions and mental health symptoms, with such information being provided to patients in an automated manner via system 100.

The following are some example use cases of how clinicians can interact with the system 100 in illustrative embodiments.

Clinician A has a phone consultation with a patient that mentions they are worried about starting school in a few months. Clinician A, with permission from the patient, instructs the patient to install an application to collect data from the patient's smartphone, with that data being input into the system 100. The patient securely sends Clinician A their data after two months, and Clinician A then directs the input of this data into the system 100, which predicts that the patient might experience an increase in their average hourly heart rate when they start school, and shows that an increased average hourly heart rate is associated with an increase in depression and anxiety symptoms. With automated assistance from the system 100, the clinician follows up with the patient, and discusses possible mindfulness treatments to relax heart rate prior to the patient beginning school.

Therapist B has been seeing a patient over a video call who just announced a family member has a terminal illness. Therapist B remembers the last time this patient had a family member pass away, and that this patient stated they would lie in bed all day, and suffered from insomnia. The patient also developed high anxiety. Therapist B consults with the patient, and the patient agrees to give their wearable data from the past month to Therapist B. Therapist B inputs this data into the system 100 to predict if similar sleep changes may occur. With automated assistance from system 100, Therapist B discusses the system outputs, which also shows the link between sleep changes and increased anxiety, and discusses a treatment plan with the patient in their next video session.

It is to be appreciated that the particular use cases described above are examples only, intended to demonstrate utility of illustrative embodiments, and should not be viewed as limiting in any way. Automated remedial actions taken based on outputs generated by a GAN-based algorithm of the type disclosed herein can include particular actions involving interaction between a processing platform implementing the GAN-based algorithm and other related equipment utilized in one or more of the use cases described above. For example, outputs generated by a GAN-based algorithm can control one or more components of a related system. In some embodiments, the GAN-based algorithm and the related equipment are implemented on the same processing platform, which may comprise a computer, a mobile telephone, a wearable device, a handheld sensor device or other type of processing device.

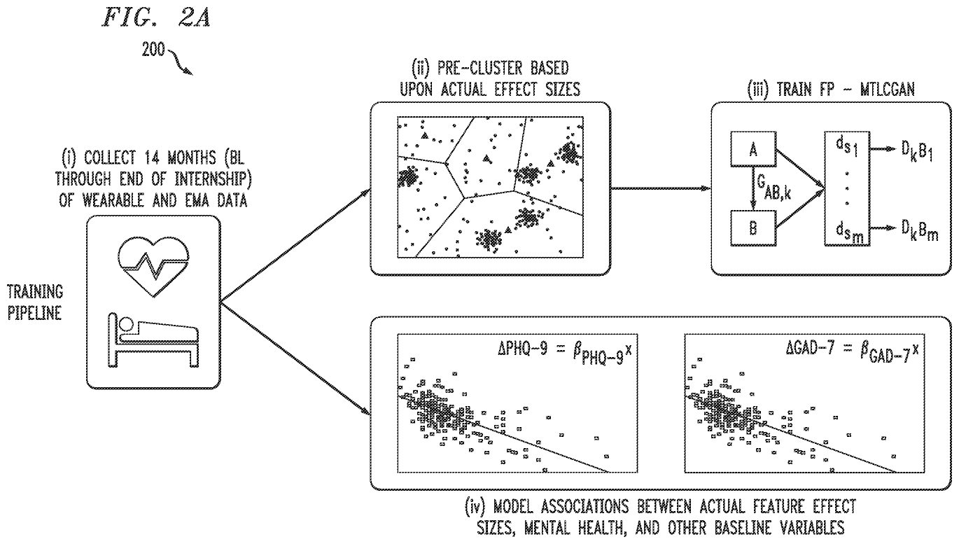

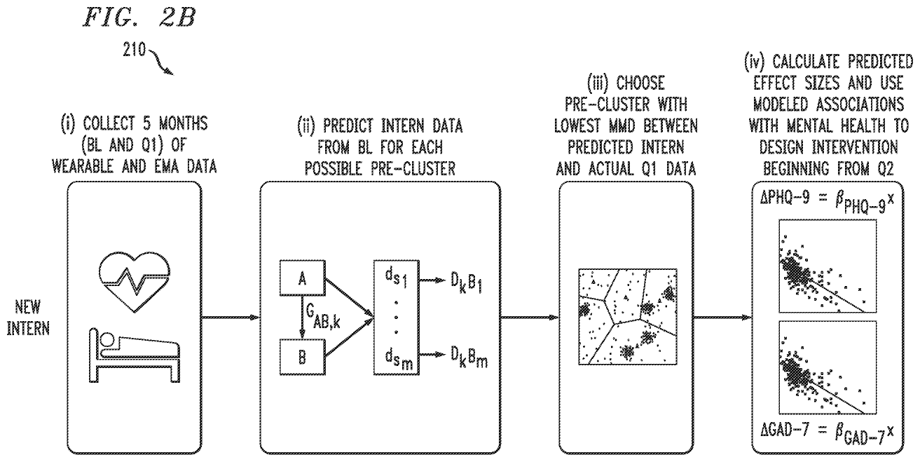

Additional details of illustrative embodiments will now be described with reference to FIGS. 2 through 10.

Illustrative embodiments utilize GANs to predict indicators of resilience using data collected from smartphone and wearable sensors. Such devices are examples of interactive, mobile, wearable, and ubiquitous technologies (IMWUT), and it is to be appreciated that a wide variety of such devices can be used in the embodiments disclosed herein. Some embodiments more particularly create a model with an interpretable loss function and use clinical metrics (e.g., Cohen's dₛ) to assess model performance.

Some embodiments to be described below more particularly predict indicators of resilience for resident physician interns, although it is to be appreciated that the disclosed techniques can be applied in numerous other contexts.

Individuals experiencing prolonged stress in the workplace are at risk for an increase in depression and anxiety symptoms. Predicting indicators of resilience, defined by sustained mental health under stress, creates the opportunity for intervention prior to a decline in mental health. As will be described, illustrative embodiments herein provide novel machine learning models using GANs to predict changes in features derived from wearable sensors and smartphone initiated ecological momentary assessments (EMAs). Specifically, we focused on predicting changes that occurred when individuals began a yearlong medical internship. We found significant positive correlations (α=0.05) between the predicted and actual feature changes using models trained on data collected three months into the internship. In addition, we demonstrated the technical feasibility of using the predicted values as indicators of resilience, by analyzing their associations with changes in depression and anxiety symptoms.

Individuals encounter a variety of stressors within the workplace, and navigating these stressors requires resilience. In 2015, the American Psychological Association found that 65% of Americans believed work to be one of the top two stressors within their lives. In addition, those who work in psychologically demanding environments are more likely to develop depression, anxiety, and substance abuse disorders. Under prolonged stress, most individuals do not experience mental health changes, showing resilience. Understanding predictors of resilience prior to mental health symptoms worsening would allow for early intervention and potentially prevent the detrimental effects of prolonged stress.

Resident physicians, both employees and trainees, are an example of a group that work within a psychologically demanding environment. This demand may contribute to higher rates of depression (25-33%) among resident physicians compared to graduate students overall and other young adults within the general population (8-15%). Introducing targeted interventions that increase resident physicians' resilience early-on in their programs could offset the potential decline in mental health that occurs after prolonged stress.

Illustrative embodiments disclosed herein can be configured to provide digital phenotypes of resilience by measuring the relationships between changes in behavior, mental health, and well-being data collected from smartphones and wearables when an individual is under stress.

In some embodiments, machine learning algorithms are implemented using GANs, a specific type of deep generative model. The algorithms were designed to predict changes in features derived from wearable sensors and self-reported well-being measures that occurred when individuals began a medical internship, which is the first year of a residency program. We also examined the technical feasibility of using the predicted changes as indicators of mental health changes that occurred when individuals adapted to internship stress. Illustrative embodiments are thus configured to predict indicators of resilience, where resilience is defined as the ability to adapt to stress, although a wide variety of other types of predictions can be made in numerous other contexts using the techniques disclosed herein.

It is expected that illustrative embodiments for predicting indicators of resilience for medical interns, employees and other individuals will provide appropriate privacy protections for the collected data and generated predictions.

In some embodiments, GANs are configured to integrate MTL with an interpretable loss function to predict changes in behavioral, physiological, and well-being features derived from wearable sensors and self-reported EMAs.

Such models are applied to predict individual-level feature changes that occurred when individuals began a medical internship.

In addition, the technical feasibility of using the predicted feature changes as resilience indicators is examined by analyzing their associations to mental health changes that took place when individuals began their internship.

Resilience, Well-being, and Mental Health

Resilience can be described as a process in which individuals positively respond or adapt to circumstances within their lives. Traditionally, when defining resilience, circumstances imply an adverse event, or negative life circumstance, that requires some amount of adjustment within an individual. That being said, resilience can be applied to circumstances that individuals face day-to-day, rather than a specific adverse event, and also many events that are traditionally viewed positively (e.g., marriage, a job promotion, beginning school) might require some amount of resilience. Resilience also implies that individuals adapt positively to the circumstances they face, which requires context dependent indicators to describe whether individuals are resilient within a specific situation. Taken together, resilience can be studied within many situations, but researchers need to be careful about how they define positive adaptation so that the implications are appropriate to the circumstances being studied.

The relationship between resilience and well-being depends upon how one chooses to measure resilience. Well-being is a construct that tries to capture an individual's internal feelings that they are flourishing, or have high life-satisfaction. Well-being is related to mental health, where mental health can subsequently be described as symptoms of well-being that capture cognitive and social functioning, and measures of well-being correlate with clinical measures of mental health.

There are multiple proposed methods to measure the relationship between resilience, mental health, and well-being. Trait resilience, which describes resilience as a personality trait that helps individuals adapt to circumstances, can be measured using a variety of rating scales, and the outcomes of these scales have correlated with mental health and well-being outcome measures. Resilience can also be described as an outcomes-based process (process resilience) that occurs when individuals adapt to minimize the impact of stress.

Resilience during a stressful life event can significantly minimize mental health changes, and thus researchers typically characterize individuals who do not experience mental health changes in response to stress as resilient. In some embodiments, we use changes in mental health and well-being that occurred under stress as a measure of resilience. It is important to note that there are a variety of environmental factors that could confound our choice of mental health changes as a resilience measure, but finding an accurate measure of resilience is still an ongoing research topic. We also focused in some embodiments on predicting resilience within a specific stressful situation (i.e., stress resilience). This methodology should not be confused with predicting trait resilience or broader indicators of process resilience. For convenience, we will use resilience, stress resilience and process resilience interchangeably throughout the rest of the description, unless otherwise specified.

Resident Physicians and Mental Health Resident physicians, part employee and part trainee, are a specific population that experience a variety of situational, personal, and professional stressors throughout the duration of their programs. After prolonged occupational stress, residents experience burnout, which is described by emotional exhaustion, cynicism, and a sense of self inefficacy. Burnout is dangerous for resident physicians' mental health, and is associated with increased depression and anxiety. The global COVID-19 pandemic has exposed the importance of resident physicians' mental health for sustaining a clinical workforce. By measuring residents' mental health throughout the duration of their programs, researchers could potentially find indicators of resilience and identify residents that require support.

Resident physicians are particularly at risk for changes in mental health during the first year of their program, often called a medical internship. During the medical internship, residents often experience an increase in depression, anxiety, fatigue, and distress that can persist throughout the duration of their residency programs. There are a number of baseline factors that are associated with changes in mental health and well-being during an internship, and behavioral changes that occur during the internship may be indicative of future mental health changes. Creating resilience-building programs or other types of interventions to increase intern resilience at the beginning of a residency program can reduce the impact of stress on mental health and/or reduce sustained declines in mental health.

Illustrative embodiments herein create unobtrusive measurement systems that can detect resilient behaviors early-on so as to help residents identify mental health risk factors and take action before symptoms develop. This data could also be anonymized and aggregated to guide program directors towards structural interventions (e.g., increased schedule flexibility) that improve mental health.

Such embodiments address significant resident concerns with more obtrusive arrangements. For example, residents may choose not to engage in certain interventions to improve resilience and mental health, citing that they do not have time or access to treatment, they would prefer to self-manage their mental health, and they are concerned about the confidentiality and potential social consequences of seeking external treatment (perceived stigma). Illustrative embodiments disclosed herein address and overcome these concerns, thereby facilitating deployment of prediction-driven interventions that can improve resident mental health and well-being.

In some embodiments, we predict behavioral, physiological, and well-being feature changes that occurred when individuals experienced novel stress from beginning a medical internship. Additional considerations that should be explicitly defined are the ethical and privacy standards for the collection of resident physician data, and employee data more broadly. The need to create these standards is more urgent when the collected data can be used to predict employee mental health and well-being. As mentioned elsewhere herein, it is generally desirable that prediction-driven intervention or other types of remediation relating to employee mental health be codesigned with employee input to ensure that appropriate ethical and privacy requirements are established.

Predicting Mental Health and Well-being Using Passive Sensors and Ecological Momentary Assessments (EMAs)

Passive sensing along with EMAs delivered through a smartphone application (e.g., smartphone sensing) can be used to predict trajectories of mental health and well-being. A passive sensor is any sensor that can collect data with little-to-no human interaction. EMAs are in-the-moment assessments, often delivered digitally, that are used to collect more frequent measurements of mental health outside of a clinic. The StudentLife and SNAPSHOT studies collected data from smartphone sensors and EMAs to find significant correlations between the data collected and the mental health and well-being of students. These technologies can also be used to predict trajectories of serious chronic mental illness, including bipolar disorder, schizophrenia, and depression. Thus, there is a wealth of evidence that behavioral data collected by passive sensors combined with EMAs can predict individuals' mental health and well-being.

Wearable technologies, such as Apple Watches and Fitbits, are devices equipped with passive sensors that could be used to monitor mental health and well-being. Sensors embedded in wearable devices can collect both activity and physiological data. Wearable and smartphone sensing features have been combined to predict mental health, and are being used to measure workplace performance, psychological traits, and physical characteristics (e.g., sleep, activity). Previous analysis using Fitbit data from the Intern Health Study, which is the primary dataset used in some embodiments herein, found that behavioral data collected from the Fitbit can be used to predict daily mood EMAs. In some embodiments, we use Fitbit and EMA data collected during the Intern Health Study to predict changes in behavioral, physiological, and well-being features that occurred when individuals began a medical internship. We then studied how these changes were associated with changes in mental health that occurred under stress, which can be used as an indicator of resilience.

Generative Models

In some embodiments, we predict changes in behavior, physiology, and well-being as a result of starting a medical internship, using a set of passive sensing features and EMA. To complete this task, we generate a during-internship distribution of behaviors and well-being features for each individual using a baseline distribution of the same features collected prior to the start of the internship. We use a generative model to generate a distribution from a given input baseline distribution.

Generative modeling is a branch of machine learning used to model multivariate probability distributions over a set of features, and deep generative models (DGMs) apply deep neural networks as a framework for generative modeling. DGMs often rely on optimizing approximations to intractable likelihoods. GANs in some embodiments are implemented as a type of DGM that can generate high quality samples without the need for optimizing a likelihood-based objective. Modifications of the original GAN framework have continued to improve GAN sample quality.

Researchers have extended the GAN framework to be able to generate samples conditioned on producing a specific output. A conditional GAN (CGAN) introduces a structured loss to try and generate a specific image conditioned on a label, text, or to transfer the style of an image but still retain characteristics of an input image. GANs are difficult to train as they suffer from the problem of mode collapse, where a GAN learns to sample from a small area of the true distribution that locally minimizes the model objective. This problem can become exaggerated within more complicated distributions, and makes GAN usage problematic within multivariate datasets. Some applications of GANs to multivariate data have used more complex normalization techniques to try and help the generator learn multimodal, multivariate distributions. In some embodiments, we provide a novel formulation of the CGAN with an interpretable loss function to generate feature changes that occurred when individuals began a medical internship.

Multi-Task Learning (MTL)

We used a GAN to model changes in multivariate data, where not only the individual features within the multivariate data come from different distributions, but feature distributions collected from different participants can vary. Within classical machine learning, MTL is a popular method for training a single machine learning model to learn multiple objectives. Traditionally, MTL is viewed as a regularization technique that constrains multiple machine learning objectives by a shared set of model parameters. Yet, when the machine learning objectives are similar and data is scarce, MTL can be used to share meaningful information across multiple learning objectives. This approach to MTL has been applied to problems with features derived from sensor data, specifically to train separate objectives that relate to separate feature outcomes (e.g., predicting mood versus stress), or to train separate models for participants that followed different behavioral patterns within the same prediction task.

Deep generative models have incorporated MTL. For example, a multi-task variational autoencoder has been configured to learn both a binary classification and multi-class classification task simultaneously. As another example, MTL has been applied to GANs for learning disentangled feature representations within images. In some embodiments disclosed herein, we integrated MTL into CGANs, and examine if using MTL improved CGAN performance on complex, multivariate feature distributions derived from wearable sensors and EMA.

Example Intern Health Study and Dataset

Some embodiments herein utilize data collected as part of an Intern Health Study to predict indicators of resilience, although it is to be appreciated that other data sets can be used in other embodiments. The Intern Health Study referred to herein is an ongoing multi-site prospective cohort study to understand the links between behaviors, mental health, and well-being as resident physicians adapted to the stress of their programs. The first year of residency, also called a medical internship, is known to impact resident mental health and well-being. Participating sites were located across the United States, and a full list of participating sites can be found on the study websites.

Interns starting their residency at a participating site were eligible to enroll online. After consenting to the study, participants were mailed a Fitbit Charge 2 for passive behavioral and physiology tracking, and completed a baseline assessment via an installed smartphone study application 1-2 months prior to the commencement of the internship. In addition, the study application sent notifications to complete daily mood EMAs, and facilitated data transfer from the Fitbit to a secure storage platform. Participants were asked to participate in Fitbit tracking and complete daily EMAs beginning 1-2 months prior to their internship through the end of the internship (˜14 months total). Lastly, participants completed quarterly mental health and well-being assessments at internship months 3, 6, 9, and 12, to further gauge how they were adapting to their new work. Table 1 describes the passive sensing and EMA data and Table 2 describes the baseline and quarterly assessment data collected.

This study was approved by the University of Michigan Institutional Review Board (IRB) and all subjects provided informed consent after receiving a complete description of the study. The collected data was used for research purposes only. In addition, data collection for the Intern Health Study was funded by the National Institute of Mental Health (RO1 MH101459, American Foundation for Suicide Prevention), and the University of Michigan Depression Center and the Taubman Medical Institute.

Passive Wearable Sensing

As indicated above, participants were mailed a Fitbit Charge 2. The Fitbit device continuously tracked minute-by-minute step counts, heart rate, recorded whether a participant was sleeping, and the type of sleep. Prior research has examined and determined that Fitbit's are an accurate consumer product for tracking sleep, activity, and heart rate for research purposes. Information about how Fitbit devices track heart rate, infer sleep states, and step counts is available on the Fitbit website, but is limited due to the proprietary nature of Fitbit's algorithms. We will briefly describe what is known below.

Fitbit uses a three-axis accelerometer to infer step count information. To detect heart rate, LED lights installed on the bottom of the Fitbit flash many times per second, and light-sensitive photodiodes then detect volume changes within wrist capillaries to infer heart rate beats per minute (BPM). Lastly, Fitbit combines the accelerometer and heart rate information to infer when an individual is sleeping, by measuring when an individual has stopped moving for one hour, and then measuring changes in heart rate to infer the sleep stage. The Fitbit application programming interface (API) outputs two different sleep categorizations, and a query to the API may respond in a mix of the two categorizations. The classic categorization uses the accelerometer to infer general sleep categories (e.g., asleep, restless), and the newer stages categorization uses the accelerometer and heart rate monitor to infer sleep stages (e.g., deep, light, rapid eye movement). The Fitbit also collects data on short wake cycles (e.g., <3 minutes) that occur between sleep.

Mood EMA

As noted above, EMAs are a standard method for assessing in-situ mental health and well-being. EMAs were completed daily by participants through the study smartphone application. The EMA contained one question that asked participants to rate their daily average mood from 1 (low) to 10 (high).

TABLE 1
Passive sensing and EMA data collected during the Intern
Health Study through the FitBit and study application.

| Data Type | Description |
|---|---|
| Heart rate | Heart rate each minute |
| Steps | Step count each minute |
| Sleep | The duration of sleep and short wake cycles, when the |
| | sleep event was recorded, as well as the category |
| Mood EMA | Question prompt: On a scale of 1 (low) to |
| | 10 (high), what was your average mood today? |

Baseline and Quarterly Assessments

Participants completed baseline (BL) and quarterly (Q1-4) assessments upon beginning their internship that contained questions regarding demographics (e.g., sex), medical specialty, personality traits (e.g., neuroticism), mental health, and life events. The assessments were delivered through the study smartphone application. Only a subset of the assessments were used in illustrative embodiments herein. These assessments are listed in Table 2. The mental health assessments included the nine question patient health questionnaire (PHQ-9) for depression and seven question generalized anxiety disorder (GAD-7) measure. Neuroticism was assessed using the NEO-Five Factor Inventory, and early family environment was assessed with the Risky Families Questionnaire. Some embodiments herein did not use the demographic variables or specialty information. However, other embodiments disclosed herein utilize demographic variables and/or specialty information.

TABLE 2
Assessments collected during the Intern Health
Study and used in illustrative embodiments.

| Measure | Frequency | Description |
|---|---|---|
| Neuroticism | Once at baseline | NEO-Five Factor Inventory |
| Stressful Life Events | Once at baseline and quarterly | Have you had at least one |
| | throughout internship | stressful life event occur during |
| | | this period (e.g., BL)? |
| Early Family Environment | Once at baseline | Risky Families Questionnaire |
| Depression | Once at baseline and quarterly | Nine Question Patient Health |
| | throughout internship | Questionnaire (PHQ-9) |
| Anxiety | Once at baseline and quarterly | Seven Question Generalized |
| | throughout internship | Anxiety Disorder Measure |
| | | (GAD-7) |

Intervention

An intervention was delivered beginning with the 2018 cohort of the Intern Health Study to improve weekly mood, physical activity, and sleep. Participants within the intervention group were randomly assigned each week to receive push notifications related to a particular category (e.g., mood, activity, sleep, or no notifications). In some embodiments, we did not focus on the intervention results, and found through a brief analysis that the intervention did not affect our predictions.

Example Prediction System Using GANs

An example prediction system utilizing GANs to predict indicators of resilience in the context of a medical residency internship will now be described in more detail.

FIGS. 2A and 2B show respective training and testing processes utilized in the example prediction system. Such a system and its associated training and testing processes are illustratively implemented in the processing platform 102 of FIG. 1, or in other arrangements of one or more processing devices. FIG. 2A more specifically illustrates a training process 200 having steps (i) through (iv) as shown, and FIG. 2B more specifically illustrates a testing process 210 showing how one or more trained models from the training process 200 are deployed to generate predictions in the testing context. The baseline (BL) period is defined as the two month period before the internship (the INTERN period), and the quarterly periods (Q1-4) refer to each three month period within the yearlong internship.

In the training process 200 of FIG. 2A, we collect 14 months of data from medical interns in step (i), use this data in step (ii) to create clusters (tasks) for participant MTL models, and then train models in step (iii) that generate during-internship multivariate passive sensing and EMA densities per participant. We also model associations between actual feature effect sizes, mental health, and other baseline variables, as shown in step (iv).

In the testing process 210 of FIG. 2B, we collect data through the first quarter of the internship in step (i), and use this data to generate a multivariate density of passive sensing and EMA data per participant for the entirety of the internship in step (ii). If the trained model uses participant MTL, we generate data in step (ii) for each cluster (task), and in step (iii) we choose the cluster whose generated data most closely matches the actual Q1 data of the participant. We also calculate predicted effect sizes and use modeled associations with mental health to design intervention beginning from Q2, as indicated in step (iv).

Feature Creation

Features were created from the Fitbit passive sensing and EMA data collected. We now briefly describe each feature in more detail. A summary of features can be found in Table 3.

1. Heart Rate. The Fitbit tracks minute by minute heart rate. We computed the mean hourly heart rate for each hour and participant. We chose to use the mean instead of the median as a summary feature in some embodiments, by way of example, as extreme heart rate values recorded within an hour would be captured by using the mean. Heart rate variability, which can be used as an indicator for stress, was not available for all participants within our example dataset, and was not used in illustrative embodiments. However, heart rate variability as well as numerous other additional or alternative features can be used in other embodiments.

2. Daily Mood EMA. Participants were notified to complete a daily mood EMA through the smartphone application. Since the mood EMA was recorded daily, we interpolated the EMA to create an hourly feature using the following procedure. If a mood EMA was completed on a day, we filled the hours of that day with the EMA value, from the time the participant woke up from a previous sleep cycle, up to the time when the participant woke up following the next sleep cycle that was greater than two hours. If multiple mood EMAs were recorded on a day (implying the participant completed the survey more than once), the average of the mood EMAs was taken, and this average value was used for interpolation. Mood EMAs were filled up until 24 hours after the EMA was completed, assuming another mood EMA was not completed on the following day. Lastly, we added random noise ε˜Uniform (0, 0.2) to each mood EMA so that we could model mood as a continuous variable. Because the hourly mood feature is effectively an interpolated daily mood EMA, we will refer to this feature as a daily mood EMA in the following description.

3. Sleep. The Fibit categorizes the type of sleep, as described previously herein, and records short wake cycles that occur in-between sleep. For simplicity, we created two hourly sleep variables, indicating the total number of seconds of sleep (within any type of sleep), and the total number of seconds in bed (which includes both sleep and short wake cycles).

4. Steps. The Fitbit tracks minute by minute step counts. We summed through all steps taken within an hour to create an hourly step count feature.

TABLE 3
Passive sensing and EMA features
used in illustrative embodiments.

| Data Type | Derived Feature |
|---|---|
| Heart rate | The hourly mean heart rate |
| Mood EMA | Interpolated daily self-reported EMA |
| Sleep | Time (in seconds) spent sleeping and in bed over an hour |
| Steps | Number of steps taken over an hour |

Data Cleaning and Inclusion Criteria

After creating the initial hourly features from raw data, we analyzed the data for missing values and outliers. The following types of missing data were identified, with mitigation procedures.

For step and sleep features, we identified hours that contained classified sleep, but no recorded steps, and vice-versa. Missing data for step and sleep features were filled with Os during hours where either of these cases occurred, indicating our belief that the Fitbit did not collect sleep data when a participant was awake and active, and did not collect step data when a participant was asleep.

After creating the interpolated mood EMA, we dropped all remaining hours missing an interpolated mood EMA.

Heart rate data should be continuously recorded by the Fitbit. We dropped hours that did not contain any heart rate data for a participant.

After dropping missing values, we filtered out outliers. Outliers were filtered using an Isolation Forest algorithm. Isolation forests recursively partition data through randomly selected features. A set of partitions can be described as a path to a set of samples, and samples that are partitioned by shorter paths are classified as outliers. We created an Isolation Forest using the scikit-learn library, with 250 trees, and randomly partitioned samples into each tree. The maximum number of features per tree was set to the length of the feature space. 73,722 samples (2.9% of the total samples) were classified as outliers, and removed. After all data cleaning was completed we filtered out study participants that did not have at least 100 total hours of data prior to the internship starting, and during the internship year. We lastly filtered out individuals who had an hourly feature variance of zero, for any hourly feature. A summary of the data filtering procedure can be found in Table 4.

In some embodiments, the objective is to predict indicators of resilience when individuals were introduced to the novel, prolonged stress of a medical internship. Ideally, we would have predicted changes that occurred at each quarter of the internship and developed a more fine-grained notion of when individuals were resilient. As the internship progressed, the availability and quality of participant data, specifically after the second quarter of the internship (see Table 4), decreased. For example, after data cleaning, hourly data did not remain for many participants during Q3 and Q4. Thus, we focused on a simpler task, which was to predict feature changes that occurred before and after individuals began the internship.

TABLE 4
Overview of the data filtering process, including the number
of participants and median and interquartile range (IQR)
for hours of data, split by baseline (BL) and each quarter (Q1-4). The
median is the 50^{th} percentile, and the IQR is a range representing
the (25-75^{th}) percentiles of the data. Note that we enforced
participants to have 100 hours of collected data in BL, and
within the combined Q1-4, hence the median increase in hours
of data for BL after data cleaning.

| | | Before Cleaning | | After Cleaning | |
|---|---|---|---|---|---|
| Number of Participants | | 2,668 | | 775 | |
| Hours of Data | BL | 307 | (28-634) | 352 | (227-529) |
| | Q1 | 850 | (20-1,850) | 785 | (390-1,197) |
| | Q2 | 875 | (60-1,867) | 400 | (73-949) |
| | Q3 | 449 | (18-1,667) | 0 | (0-515) |
| | Q4 | 551 | (20-1,719) | 135 | (0-637) |

Modeling Approach

We developed DGMs to predict the during-internship joint feature distribution from data collected prior to the internship. Predicting individual-level feature changes that occurred when individuals experienced prolonged stress from data prior to the stressor allows us to develop interventions to increase resilience and offset potential negative mental health and well-being changes.

For the following description, we will refer to the period prior to the internship as the baseline (BL) period, and the during-internship period as the internship (INTERN) period. For a set of m features, we will define a BL data point as a∈A, a∈ [symbol] ^{m} and an INTERN data point as b∈B, b∈ [symbol] m. Lastly, a predicted INTERN data point will be defined as b′∈B′, b′∈ [symbol] ^{m}.

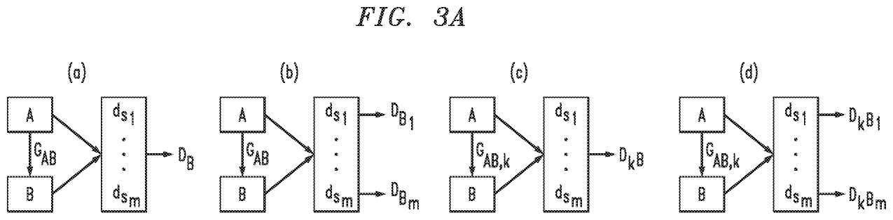

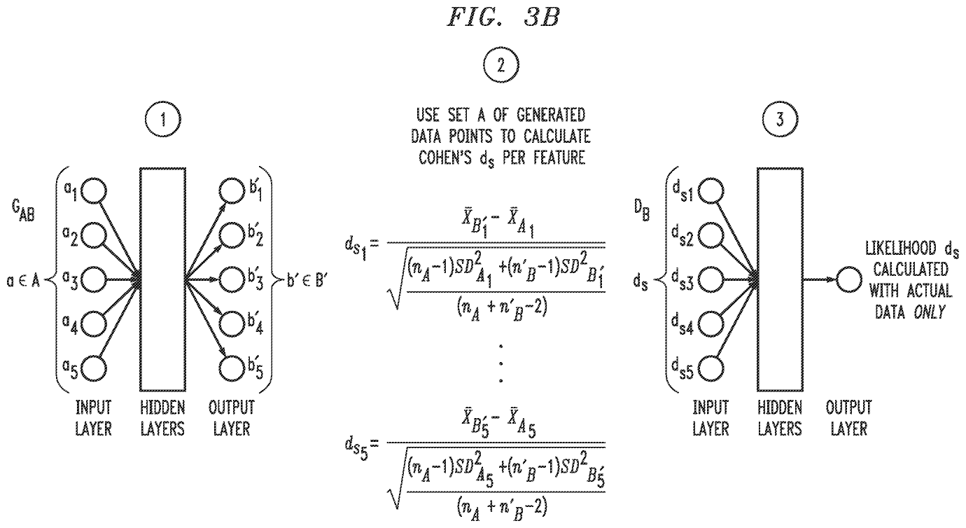

FIG. 3A shows example configurations of GANs used in GAN-based algorithms in illustrative embodiments. More particularly, the four different parts of the figure show four different example GAN models, namely, (a) CGAN, (b) F-MTLCGAN, (c) P-MTLCGAN and (d) FP-MTLCGAN, where MTL denotes multi-task learning, F denotes feature and P denotes participant.

FIG. 3B shows a more detailed view of an example CGAN architecture shown in part (a) of FIG. 3A. Hidden layers are shown as black boxes for simplicity, but they are composed of multiple fully connected neural network layers. As an example, we describe how a multivariate data point can be generated and passed into the discriminator. In step (1), a single multivariate baseline hourly data point a E A is input into the generator, G_{AB}, which outputs a single generated multivariate hourly internship data point, b‘ E B’. After inputting a set of multivariate baseline data points into the generator and outputting a set of generated internship data points for an individual in step (1), a generated internship multivariate mean X_{B}, and sample standard deviation SD_{B,j} can be calculated in step (2) for each feature, j,j∈{1, . . . , m}, where there exist m total features. This can be used to then calculate, also in step (2), a predicted Cohen's dₛ for each feature, dₛ_{₁}, . . . , dₛ_{ₘ}. In step (3), the Cohen's dₛ for each feature can be input into the multivariate input layer of the discriminator D_{B}, which outputs the probability the multivariate Cohen's dₛ was calculated using actual or generated data. For the feature MTL networks, the discriminators for each feature's Cohen's dₛ_{ⱼ} do not share any layers. For the participant MTL networks, there are additional output layers specific to each cluster on the generator, and each cluster has a unique discriminator which does not share any layers with other cluster discriminators. A GAN can be characterized as implementing a two-player game between generator and discriminator neural networks that compete with each other, resulting in increasing sample quality. The operation of the GAN will now be described in more detail, starting with the classical GAN framework, and then expanding upon this framework until we have reached the modeling approach used in some embodiments herein. Let z∈Z, z∈ [symbol] be a random noise scalar sampled from a standard normal distribution. Let G: [symbol] → [symbol] ^{m} be a generator, or in other words, a neural network that generates a synthetic vector a′∈A′, a′∈ [symbol] ^{m} from a random z, a′=G(z). Let D: [symbol] ^{m}→ [symbol] be discriminator, or an additional neural network that tries to distinguish a generated point a′ from an actual data point a. The discriminator outputs a probability that a point comes from the actual data distribution, and thus a perfect discriminator would output D(a)=1 and D(a′)=D(G(z))=0. We can thus define the GAN objective and loss as follows:

max_{G}min_{D} [symbol] _{GAN}(G,D,A,Z) [symbol] _{GAN}(G,D,A,Z)= [symbol] _{a∈A}[log(1−D(a))]+ [symbol] _{z∈Z}[log D(G(z))]

The generative models used in some embodiments are based upon a CGAN, as illustrated in part (a) of FIG. 3A, and shown in more detail in FIG. 3B. Specifically, the CGAN generated b′∈B′ from α∈A. The generator was a fully connected neural network that input a BL data point and generated a synthetic INTERN data point. Specifically, we defined generator, G_{AB}: [symbol] ^{m}, [symbol] ^{m}, such that b′=G_{AB}(a). We also defined the discriminator D_{B}: [symbol] ^{m}→ [symbol] . The CGAN objective was the following two-player game with a mean-squared error (MSE) loss. The MSE loss was found to be more stable during training within similar contexts. A binary cross-entropy (BCE) loss was also used during our initial network formulation, but we found the MSE loss produced better results. The CGAN objective and loss were:

max_{G}_{_{AB}} min_{D}_{_{B}} [symbol] _{GAN}(G_{AB},D_{B},A,B)

[symbol]

_{GAN}(G_{AB},D_{B},A,B)= [symbol] _{b∈B}[(D_{B}(b)−1)²]+ [symbol] _{a∈A}[D_{B}(G_{AB}(a))²]

We also introduced a conditional loss, or L_{CON}, to model the differences between the actual b∈B and generated b′∈B′. In some embodiments, specific data points a∈A and b∈B are unpaired in the sense that there is no specific a that should directly map to a b. That being said, a well-generated distribution for an individual, B′, should have the same characteristics (e.g., mean, variance, skew) as the actual distribution B. We thus choose a conditional loss function focused on high level distribution characteristics instead of trying to minimize the error between individual generated and actual data points.

The maximum mean discrepancy (MMD) is a two sample test, testing the hypothesis that two samples are drawn from the same distribution. The MMD has been integrated into the GAN framework as a GAN loss, but we applied the MMD as a conditional loss. The MMD compares the differences between a kernel estimated over the individual actual and generated distributions, and the mixed distribution of actual and generated data. The MMD approaches 0 as the sum of the individual kernels approach the mix (i.e., the distributions become equivalent). Again, let a∈A be a set of data points used to generate b′∈B′. Let k(x, y) be a kernel function. We can define the MMD conditional loss as follows:

L_{CON}(G_{AB},A,B)= [symbol] [k(G_{AB}(a),G_{AB}(a*))+k(b,b*)−2k(G_{AB}(a),b)]

We used the kernel function k(x, y)=Σ_{q=-1}^{K}k′_{σ}_{_{q}} where k′_{σ}_{_{q}}(x, y) is a radial basis function (RBF) and σ_{q} is an adjustable bandwidth parameter. We let σ_{q} equal {1, 2, 4, 8, 16}.

The full objective and loss can be thus described as

max_{G}_{_{AB}}min_{D}_{_{B}}L(G_{AB},D_{B},A,B) L(G_{AB},D_{B},A,B)=L_{GAN}(G_{AB},D_{B},A,B)−L_{CON}(G_{AB},A,B)

In addition to the MMD loss, we experimented with absolute mean error and squared mean error conditional loss functions, but found the MMD loss outperformed these other conditional losses. Although illustrative embodiments disclosed herein utilize models trained using the conditional MMD loss, other loss measures can be used.

Additional details of the effect size discriminator will now be provided. We created a novel CGAN framework specifically for modeling behavioral, physiological, and well-being feature changes. To do this, we had the discriminator operate on an interpretable, calculated feature space, namely the feature effect sizes, or Cohen's dₛ. We expected that the raw A and B distributions would overlap, and thus having the GAN loss operate on the raw feature space would confuse the optimization algorithm. By having the discriminator operate on the feature effect sizes, we would give better feedback to the GAN loss. In addition, the GAN loss could now be interpreted: if the trained discriminator cannot distinguish between actual and predicted data points, the predicted and actual Cohen's dₛ should match. Interpretability is an important factor when creating machine learning models with healthcare applications.

We calculated the effect size, or Cohen's dₛ, per feature, and used the actual and predicted effect sizes as inputs to the discriminator networks. The Cohen's dₛ is typically used to measure the effect of an intervention in a randomized control trial (RCT) across two groups of [symbol] independent observations. For a specific feature a₁∈Aⱼ, bⱼ∈Bⱼ, a₁∈ [symbol] , bⱼ∈ [symbol] and participant, let n be the batch training size, with respective batch means X̅_{A}_{ⱼ}, X̅_{B}_{ⱼ}, and batch sample standard deviations SD_{A}j, SD_{B}j. We can calculate the effect size for a feature, dₛ, as follows:

$${d}_{{s}_{j}}^{{A}_{j} , {B}_{j}} = \frac{{\overline{X}}_{{B}_{j}} - {\overline{X}}_{{A}_{j}}}{\sqrt{\frac{{SD}_{{A}_{j}}^{2} + {SD}_{{B}_{j}}^{2}}{2}}}$$

We can also calculate the predicted effect size:

$${d}_{{s}_{j}}^{{{A}_{j} ( {G}_{AB} )}_{j}} = \frac{{\overline{X}}_{{( {G}_{AB} )}_{j} ( A )} - {\overline{X}}_{{A}_{j}}}{\sqrt{\frac{{SD}_{{A}_{j}}^{2} + {SD}_{{( {G}_{AB} )}_{j} ( A )}^{2}}{2}}}$$

The GAN loss can be re-written as the following where dₛ∈ [symbol] ^{m} is a vector of effect sizes across m features. Note that we are no longer taking the expectation over the data, since the Cohen's dₛ summarizes the baseline and generated distribution changes:

[symbol]

_{GAN}(G_{AB},D_{B},A,B)=[D_{B}(\vec{d}ₛ^{A,B})−1]²+[D_{B}(\vec{d}ₛ^{A,G}^{_{AB}})]²

Some embodiments herein utilize MTL in a CGAN. The CGAN in some embodiments is based on a CycleGAN. MTL is a machine learning technique used to train separate, but related prediction tasks together. MTL can be described as a regularizer: by training separate tasks within a single model, we reduce the total number of model parameters. MTL has been used to train separate but related facial detection tasks, such as face pose estimation and facial localization, and has also improved neural network model performance within tasks that individually have scarce data. We experimented with two novel applications of MTL within a CGAN. The two novel formulations and assumptions underlying these formulations are as follows:
(1) We assumed that data generation for each hourly feature was a separate, but related task. Training these tasks together could regularize each individual task by predicting the joint feature distribution.
(2) Participants experienced a variety of feature changes when beginning the internship, but training a model for each participant would result in overfitting, and not generalize to unseen individuals. MTL can prevent this overfitting by training individual-level models together.

As the example dataset did not include enough data to train a task for each individual, we instead clustered individuals together that experienced similar feature changes once the internship began, and treated training a model for each cluster as a separate task. We will describe the pre-clustering procedure elsewhere herein.

We considered a multidimensional data point a=(a₁, . . . , aₘ), a∈ [symbol] ^{m}, where the process to train a network to accurately generate a single aⱼ∈ [symbol] is defined as a single task. We first created a Feature Multi-Task Learning CGAN (F-MTLCGAN), illustrated in part (b) of FIG. 3A, where we utilized the CycleGAN as a base model, but then replaced the single discriminators D_{A}, D_{B} with a separate discriminator, D_{A}_{ⱼ}, D_{B}_{ⱼ}: [symbol] → [symbol] for each feature that is [symbol] trained to predict whether a feature's effect size, dₛ_{ⱼ}, is an actual or predicted effect size. The new GAN loss becomes:

$${ℒ}_{GAN} = \frac{1}{m} {\sum }_{j = 1}^{m} {ℒ}_{{j}^{GAN}} ( {( {G}_{AB} )}_{j} , {D}_{{B}_{j}} , {A}_{j} , {B}_{j} )$$

$${ℒ}_{{j}^{GAN}} ( {( {G}_{AB} )}_{j} , {D}_{{B}_{j}} , {A}_{j} , {B}_{j} ) = {[ {D}_{{B}_{j}} ( {d}_{{s}_{j}}^{{A}_{j} , {B}_{j}} ) - 1 ]}^{2} + {[ {D}_{{B}_{j}} ( {d}_{{s}_{j}}^{{A}_{j} , {( {G}_{AB} )}_{j}} ) ]}^{2}$$

We also created a Participant Multi-Task Learning CGAN (P-MTLCGAN), illustrated in part (c) of FIG. 3A, where we first clustered participants based upon their actual effect size, and treated each cluster as a single task. Specifically, we added an extra linear layer to the generator, G_{AB}, and trained a separate discriminator for each cluster. If we have K clusters, we would thus train K separate D_{B}. During training, we can pick a specific participant, and only back-propagate through the shared layers and cluster-specific layers for that participant. In this case, let k be the cluster for a participant, and let G_{AB},k, be a generator that will propagate an input through an extra linear layer specifically for cluster k. Let DₖBi be the discriminator for cluster k, and \vec{d}ₛ the multidimensional effect size. The GAN loss becomes:

[symbol]

_{GAN}(G_{AB,k},Dₖ_{^{B}},A,B)=[Dₖ_{^{B}}(\vec{d}ₛ^{A,B})−1]²+[Dₖ_{^{B}}(\vec{d}ₛ^{A,G}^{_{AB,k}})]² b∈B,a∈A,b∈ [symbol] ^{m},a∈ [symbol] ^{m},\vec{d}ₛ∈ [symbol] ^{m},k∈{1, . . . ,K}

Finally, we combined both the F-MTLCGAN and P-MTLCGAN to create a Feature and Participant Multi-Task Learning CGAN (FP-MTLCGAN), illustrated in part (d) of FIG. 3A, which first propagated a cluster-specific output for a participant, and then had separate [symbol] discriminators for each cluster and feature, DₖBⱼ. The FP-MTLCGAN [symbol] _{GAN} is:

$${ℒ}_{GAN} = \frac{1}{m} {\sum }_{j = 1}^{m} {ℒ}_{{j}^{GAN}} ( {( {G}_{AB , k} )}_{j} , {D}_{{k}^{{B}_{j}}} , {A}_{j} , {B}_{j} )$$

$${ℒ}_{{j}^{GAN}} ( {( {G}_{AB , k} )}_{j} , {D}_{{κ}_{j}^{B}} , {A}_{j} , {B}_{j} ) = {[ {D}_{{k}^{{B}_{j}}} ( {d}_{{s}_{j}}^{{A}_{j} , {B}_{j}} ) - 1 ]}^{2} + {[ {D}_{{k}^{{B}_{j}}} ( {d}_{{s}_{j}}^{{A}_{j} , {( {G}_{AB , k} )}_{j}} ) ]}^{2}$$

$${b}_{j} ∈ {B}_{j} , {a}_{j} ∈ {A}_{j} , {b}_{j} ∈ ℛ , {a}_{j} ∈ ℛ , {d}_{{s}_{j}} ∈ ℛ , k ∈ \{ 1 , … , K \} , \\ j ∈ \{ 1 , … , m \}$$

Training and Testing Procedure

All predictive models were trained with data from 80% of the study participants, and evaluation metrics using the trained models were calculated for the remaining 20% of participants. Deep generative models were built using Pytorch, and trained for 1,000 epochs. During each epoch, we iterated through all training participants once. Models were trained using the Adam optimizer with different initial learning rates (0.001, 0.0001). We trained models using a batch size n_{batch} where n_{batch}=min(nₐ, n_{b}) for each participant, defining nₐ, n_{b} as the number of data points within individual-level distributions A, B respectively.

All generators and discriminators used fully connected linear layers. Given m input features to a network, the generator architecture had three hidden layers of size (2 m, 4 m, 2 m), and the discriminator architecture had two hidden layers of size (2 m, 4 m). In addition, we trained a neural network regression (REG) model that optimized only the generator G_{AB}, to study if using the CGAN framework improved the prediction performance over a simpler model. We also applied participant MTL to the baseline regression model (P-MTLREG) to see if it improved baseline model performance. We experimented with adding more hidden layers across models, but deeper networks increased training time with minimal improvements to model performance.

Dropout (rate=0.2) was used for regularization between linear layers of the discriminator, and batch normalization was used for regularization between linear layers within the generator. All features were scaled between [−1, 1]. Rectified Linear Unit (ReLU) activation was used for the generator hidden layers and hyperbolic tangent (Tanh) activation was used for the output activation. Leaky ReLU (LReLU) layers were used for the discriminator hidden activation (negative slope=0.2), and a sigmoid layer was used for the output activation.

Pre-clustering for Participant MTL Models

To find initial participant clusters (individual MTL tasks) within the P-MTL models, we clustered the training data using K-Means and Ward Hierarchical Agglomerative Clustering. The effect sizes (feature changes) for each training participant and feature were calculated, and we used principle components analysis to reduce noise within the feature space. The silhouette score, which quantifies both the tightness of within-cluster data and the distance between adjacent clusters, was used to choose the number of components, clusters, and clustering algorithm. We varied the number of clusters from 2-10, and we added component dimensions until 99% of the variance between features was explained. After model training, we generated data from all clusters for each test participant. We then computed the MMD between the generated data, and the actual first quarter internship data for each test participant. The generated data from the cluster that achieved the lowest MMD per-participant compared to the first quarter data (Q1) were included within our results, and all other data were removed for that participant. Other embodiments can use both BL and Q1 data from new participants to facilitate usage of P-MTL models in these and other applications.

Evaluation Metrics

In addition to using the MMD as a loss function, we used the MMD to assess sample quality in illustrative embodiments. MMD is a good metric for assessing GAN sample quality, and has low computational complexity. The MMD was computed using the same formula described elsewhere herein.

Some embodiments disclosed herein also utilize a 1-Nearest Neighbor (1-NN) classifier. Given two sets of samples from an actual and generated distribution, S_{A}, S_{G}, we computed the accuracy of a leave one sample out (LOSO) nearest neighbor classifier, where true samples were given a positive label and generated samples were given a negative label. 1-NN classifiers have been described as ideal metrics for GANs, because they can detect when mode collapse, a common problem for GANs, occurs. We computed the LOSO accuracy of a 1-NN classifier for actual samples, or true positive rate (TPR), and generated samples, or true negative rate (TNR), for each study participant.

Additionally or alternatively, some embodiments utilize Absolute Discriminator Ratio Error (ADRE), as one example of an interpretable metric to assess the GAN loss performance. The discriminator outputs the probability that an input effect size came from the actual data. If the ratio between the trained discriminator output for the actual and predicted effect sizes equaled one, the discriminator is indicating that the predicted and actual effect sizes were both equally likely to have come from the actual data distribution. This should occur if the generator produced realistic data points. We thus defined the ADRE as follows, noting that for F-MTL models, the discriminator outputs were averaged across features to calculate the ADRE:

$$\vert 1 - \frac{{D}_{B} ( {\vec{d}}_{s}^{A , {G}_{AB}} )}{{D}_{B} ( {\vec{d}}_{s}^{A , B} )} \vert$$

In some embodiments, correlation between actual and predicted effects was determined. We calculated the Pearson's correlation (R²) between the actual and predicted Cohen's dₛ to provide a clinically interpretable metric of model performance. The Cohen's dₛ for model evaluation utilized a pooled standard deviation due to variations in the amount of data collected and generated for both the BL and INTERN periods. Starting with the Cohen's dₛ formula provided previously herein, let n_{A}, n_{B} be the amount of BL and INTERN data respectively. Note that (G_{AB})ⱼ can be substituted for Bⱼ to calculate the predicted effect size. The metric was as follows:

$${d}_{{s}_{j}}^{{A}_{j ,} {B}_{j}} = \frac{{\overline{X}}_{{B}_{j}} - {\overline{X}}_{{A}_{j}}}{\sqrt{\frac{( {n}_{A} - 1 ) {SD}_{{A}_{j}}^{2} + ( {n}_{B} - 1 ) {SD}_{{B}_{j}}^{2}}{( {n}_{A} + {n}_{B} - 2 )}}}$$

Associations between Effect Sizes and Mental Health

We used linear regression to assess the technical feasibility of applying the predicted feature changes within a resilience building intervention (see FIGS. 2A and 2B, step (iv) in each figure). Specifically, the regression modeled the relationship between the actual feature effect sizes and mental health changes that occurred as a result of beginning the internship. We also included other baseline assessment variables within the regression (see Table 2). If such associations existed between the hourly features and mental health, these associations could be applied to create interventions for unseen participants using the predicted effect sizes from our models. For example, if seconds of sleep showed a significant negative association with increased PHQ-9 and we predicted a new intern is likely to lose sleep after beginning the internship, that intern could be assigned a sleep coach to build resilient behaviors before depression symptoms increased.

The regression predicted the largest changes in PHQ-9 and GAD-7 that occurred throughout the INTERN period for each individual, compared to their baseline PHQ-9 and GAD-7 measures. For example:

ΔPHQ−9=\vec{β}_{ΔPHQ-9}_{^{x}}

ΔPHQ−9=maxᵢ₌₁₋₄{PHQ−9_{Q}_{ᵢ}}=PHQ−9_{BL}

Results from Example Prediction System

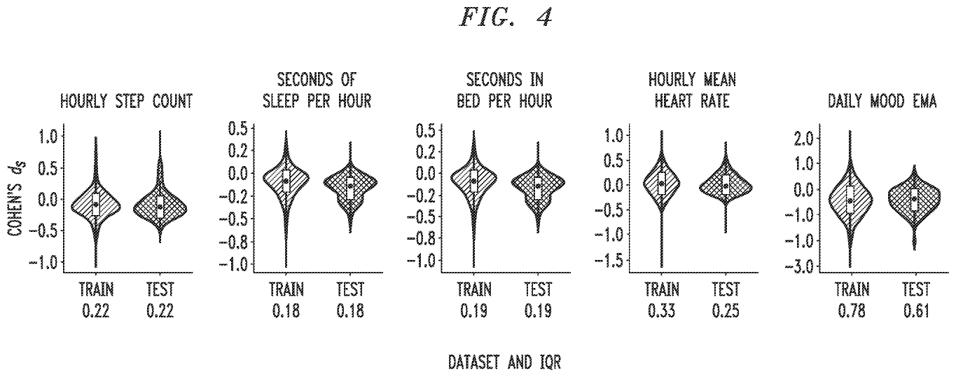

Table 5 shows a summary of the data used for model training and testing after data cleaning and outlier filtering, and FIG. 4 shows the effect size (Cohen's dₛ) distributions for each feature. More particularly, FIG. 4 shows histograms of effect sizes for training and testing data. Within each histogram shown in FIG. 4, the boxplots show the median and IQR of each effect size. The numbers below the x-ticks indicate the IQR of the Cohen's dₛ for the specified dataset. We highlighted the interquartile ranges of each feature's Cohen's dₛ, which were considerably larger in the training data for the hourly mean heart rate (0.33) and daily mood EMA (0.78) compared to the step count (0.22), seconds of sleep (0.18) and seconds in bed (0.19) per hour.

TABLE 5
Overview of data utilized to train and test
predictive models listed by median and IQR.

| | | Train | | Test | |
|---|---|---|---|---|---|
| Number of | | 621 | | 154 | |
| participants | | | | | |
| Hours of data | Baseline | 366 | (244-533) | 354 | (226-521) |
| per-participant | Internship | 1,353 | (531-2,939) | 1,542 | (699-3,341) |

Pre-Clustering

We performed a pre-clustering using the actual training data effect sizes to create each task (each cluster) for the P-MTL models. The clustering that achieved the highest silhouette score (0.32) used Agglomerative Clustering with four principle components, and resulted in two clusters with 510 and 111 training participants in each cluster respectively.

Model Performance

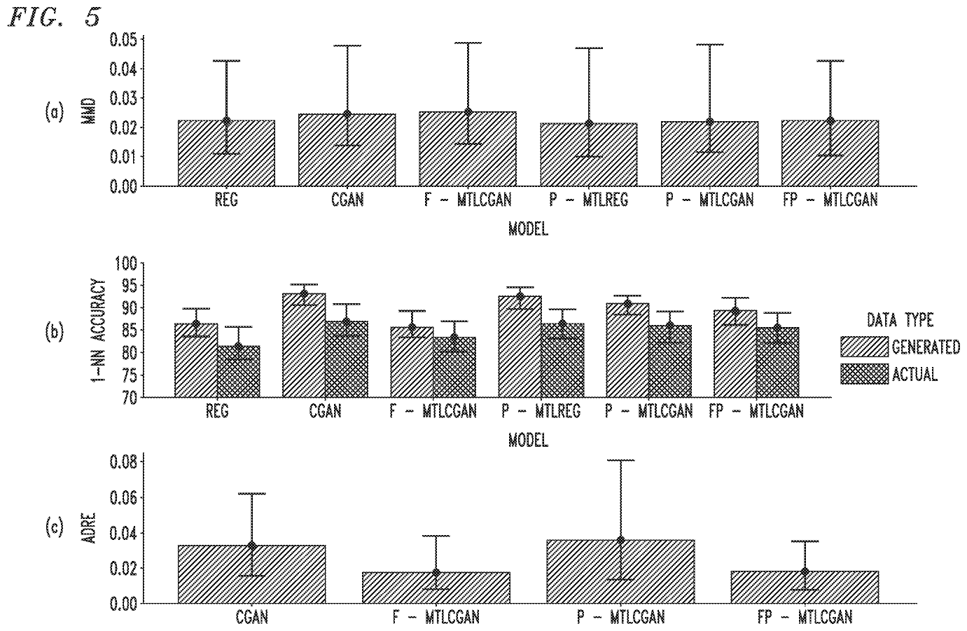

FIG. 5 shows individual model results across participants, specifically the (a) MMD, (b) 1-NN Accuracy, and (c) ADRE. The bar heights indicate the median value across participants, and the error bars indicate the IQR.

For simplicity and clarity, we have not included the results for each learning rate, and only listed the performance of the models that achieved the lowest MMD. Across participants, the FP-MTLCGAN achieved the lowest MMD (0.022, IQR 0.010-0.043). When analyzing the performance of models in the context of a 1-NN classifier, we focused on the models that achieved the lowest 1-NN Generated Accuracy. High 1-NN Generated Accuracy is an indicator of mode collapse within GANs, as high accuracy indicates the generated points are compact. The F-MTLCGAN achieved the lowest 1-NN Generated Accuracy across participants (85.6%, IQR 83.4-89.3%). Lastly, we analyzed the ADRE. The FP-MTLCGAN achieved the lowest ADRE across all participants (0.0173, IQR 0.0080-0.0352).

Individual Effect Size Correlations

We calculated the true effect size (Cohen's d^{A}^{ⱼ}^{,B}^{ⱼ}ₛ_{ⱼ}) and predicted effect size (Cohen's d^{A}^{ⱼ}^{,(G}^{_{AB}}⁾^{ⱼ}ₛ_{ⱼ}) for each test participant and feature j∈{1, . . . , m}. We then calculated the Pearson's correlation coefficient (R²) and correlation significance between the actual and predicted effect sizes. The resulting R² values are found in Table 6. The FP-MTLCGAN model had both significant (a=0.05) and relatively high correlations between all features, with R² values of (R² '² 0.62, P<0.001) for the hourly step count, (R² '² 0.69, P<0.001) seconds of sleep per hour, (R² '² 0.69, P<0.001) seconds in bed per hour, (R² '² 0.20, P<0.05) hourly mean heart rate, and (R² '² 0.23, P<0.01) for the daily mood EMA.

TABLE 6
Correlations (R²) and significance between the predicted
and actual individual-level effect sizes (Cohen's dₛ)
for each feature * P < .05, ** P < .01, *** P < .001.

| | | Seconds | Seconds | Mean | |
|---|---|---|---|---|---|
| | Step | of | in | Heart | Mood |
| Model | Count | Sleep | Bed | Rate | EMA |
| REG | 0.62*** | −0.02 | 0.01 | 0.09 | 0.09 |
| CGAN | 0.56*** | −0.05 | −0.05 | 0.20* | 0.26** |
| F - MTLCGAN | 0.62*** | −0.03 | −0.01 | 0.21** | 0.19* |
| P - MTLREG | 0.72*** | 0.68*** | 0.69*** | 0.22** | 0.10 |
| P - MTLCGAN | 0.46*** | 0.55*** | 0.55*** | 0.27*** | 0.23** |
| FP - MTLCGAN | 0.62*** | 0.69*** | 0.69*** | 0.20* | 0.23** |

Effects of MTL

We analyzed the effects of feature and participant MTL on model performance.

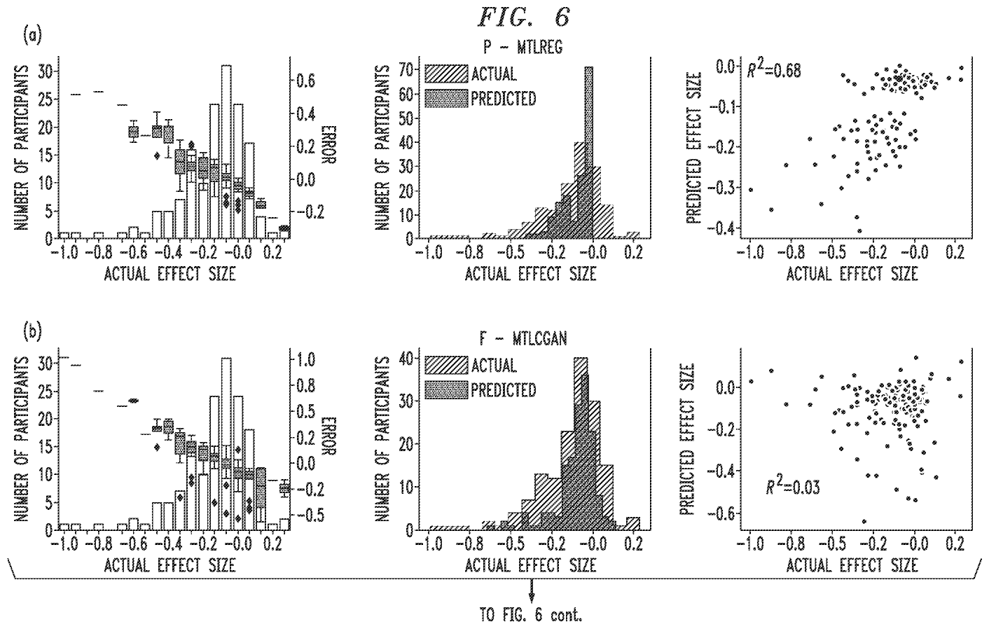

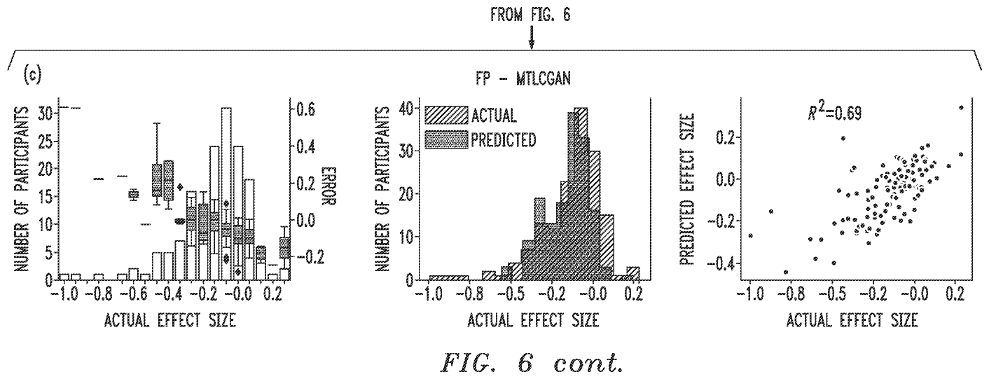

FIG. 6 shows example effect size prediction results on the test set for the seconds of sleep per hour feature, with part (a) showing results for the P-MTLREG model, part (b) showing the results for the F-MTLCGAN model, and part (c) showing the results for the FP-MTLCGAN model. The left column shows the error (predicted-actual) distributions between the individual-level actual and predicted effect sizes. The boxplots overlay a histogram describing the number of participants whose actual effect size fell into a designated range. Each box represents the error distribution for the participants within the underlying effect size range. The middle column shows a histogram comparing the actual and predicted effect sizes, and the right column shows this information in a scatterplot, where each point represents a test individual.

The results shown in FIG. 6 highlight differences in performance between the regression, CGAN, and different MTL models for the seconds of sleep feature. The left column bar charts show that all models achieved better performance around the modes of the distribution. The histograms in the middle column highlight that the CGAN models were able to predict a wider range of effect sizes compared to the regression model. The right column shows that participant MTL increased the correlation between the actual and predicted individual-level effect sizes.

Assessing the Technical Feasibility for Targeted Intervention

We created a linear regression model to examine the associations between changes in mental health that occurred as a result of the internship and the actual training effect sizes (Cohen's dₛ) per feature. If associations existed, these associations could be applied to new interns using the predicted effect sizes to create targeted resilience building interventions (see FIG. 2B). The results shown in Table 7 indicated that, while holding baseline factors constant, there were significant associations (a=0.05) within the training data between the feature and mental health changes. Specifically, for predicting changes in PHQ-9, the coefficients for the hourly step count (β=−0.08, 95% CI−0.15 to 0.00), mean heart rate (β=0.09, 95% CI 0.01 to 0.17), and daily mood EMA (β=−0.16, 95% CI−0.23 to −0.08) were all significant. For predicting changes in GAD-7, the seconds of sleep per hour (β=−1.13, 95% CI−2.23 to −0.03), seconds in bed per hour (β=1.15, 95% CI 0.06 to 2.26), mean heart rate (β=0.11, 95% CI 0.03 to 0.19) and daily mood EMA (β=−0.18, 95% CI−0.26 to −0.11) were all significant.

TABLE 7
Linear regression using the training data to show the relationships
between the effect sizes (Cohen's dₛ) and PHQ-9 and GAD-7 changes,
where * indicates a significant coefficient α = 0.05. The
horizontal line separates the effect size features and baseline variables.

| Variable | β_{ΔPHQ-9} (95% CI) | | β_{ΔGAD-7} (95% CI) | |
|---|---|---|---|---|
| Seconds of sleep per hour | −0.66 | (−1.76 to 0.44) | −1.13 | (−2.23 to −0.03)* |
| Seconds in bed per hour | 0.67 | (−0.43 to 1.77) | 1.15 | (0.06 to 2.26)* |
| Hourly steps | −0.08 | (−0.15 to 0.00)* | −0.01 | (−0.08 to 0.07) |
| Mean heart rate | 0.09 | (0.01 to 0.17)* | 0.11 | (0.03 to 0.19)* |
| Daily mood EMA | −0.16 | (−0.23 to −0.08)* | −0.18 | (−0.26 to −0.11)* |
| Baseline PHQ-9 | −0.37 | (−0.47 to −0.28)* | 0.00 | (−0.09 to 0.10) |
| Baseline GAD-7 | 0.11 | (0.01 to 0.21)* | −0.39 | (−0.49 to −0.29)* |
| Baseline stressful life events | −0.09 | (−0.17 to −0.02)* | −0.04 | (−0.11 to 0.04) |
| Early family environment score | 0.10 | (0.03 to 0.18)* | 0.03 | (−0.04 to 0.11) |
| Neuroticism | 0.30 | (0.21 to 0.39)* | 0.31 | (0.23 to 0.40)* |
| Received intervention? | 0.01 | (−0.07 to 0.08) | 0.03 | (−0.05 to 0.10) |

Illustrative embodiments described herein provide a methodology for predicting indicators of resilience, and apply this methodology to predict individual-level changes associated with beginning a medical internship. Such embodiments are focused on predicting indicators of resilience in the context of predicting feature changes that were associated with changes in mental health, and these changes occurred when individuals were adapting to novel stress. To achieve the goal of generating accurate predictions in this context, we developed novel, deep generative models with an interpretable loss function that integrated MTL into GANs. We found that our predicted feature changes positively correlated with actual feature changes that occurred among participants, and that these correlations were significant. Lastly, we showed the technical feasibility of using the predicted effect sizes to create targeted interventions for building resilient behaviors.

Predicting Resilience in the Workplace using Wearable Sensing and EMA

Mental health and well-being measurements have been used as indicators for workplace resilience. It is important to note that mental health changes related to resilience are specific to stress adaptation. One could also analyze the difference in the nature of mental health changes an individual experiences when they require resilience, compared to fluctuations in depression and anxiety symptoms that may occur within the same individual, without a specific, external stress. In addition, there are other potential measures of resilience, including assessing individuals on performance and ability to create work-life balance, that could be used to assess positive adaptation. Including such additional or alternative measures in illustrative embodiments disclosed herein could create a broader picture of whether predicted changes are specific to mental health and well-being.

Illustrative embodiments contribute to the growing research on identifying digital phenotypes. For example, the linear regression described herein identified that feature changes derived from wearable sensing and EMA were associated with PHQ-9 and GAD-7 changes. In addition, the effect sizes that were significantly associated with mental health, specifically the time sleeping and in bed, as well as the decrease in physical activity (hourly steps), are known to be associated with increased work stress, and are linked to anxiety and depression. Thus, illustrative embodiments effectively utilize passive sensing and EMA to predict indicators of resilience and to implement appropriate remediation responsive to such predictions.

Other embodiments can be configured to determine particular factors that can explain why certain individuals were resilient. For example, more data could be collected on individual-level environmental factors in order to clearly understand the link between circumstances and resilience. Nonetheless, there should be limits to how residency programs might approach identifying causes of resilience.

Implications for Residency Programs

Some embodiments herein focus on a specific type of workplace stress, namely the effects of starting a medical internship. Most research on physician mental health is focused on depression, anxiety, and burnout specifically. Illustrative embodiments utilized a dataset that included measures before and after the start of the internship to accurately predict indicators of resilience, although other types of datasets could be used.

In illustrative embodiments, we performed a pre-clustering of the training data using the Cohen's dₛ for the participant MTL models. The smaller of the two clusters held n=111 participants, accounting for 17.9% of the training data. Though the clusters were not explicitly formed using PHQ-9 and GAD-7 scores, we noticed that the smaller cluster size was comparable to nationally reported statistics for any anxiety disorder among young adults (22.3%) and a meta-analysis of moderate to severe depression among resident physicians (2.9%, 95% CI 17.5%-24.7%).

Predictions generated using the techniques disclosed herein can be used to introduce interventions to support individuals at the beginning of their internship. For example, resilience training programs have been introduced for physicians to reduce burnout that focused on promoting self-care, self-reflection, and value alignment within the workplace. A 2019 meeting convened by the Accreditation Council for Graduate Medical Education (ACGME) identified that having in place “systems to prevent and respond to distress and mental health problems experienced during residency” help resident physicians manage times of difficulty. Illustrative embodiments can utilize GAN-based predictions to determine particular remediation actions for particular subjects.

In addition, by identifying specific behavioral, physiological and well-being profiles that were related to mental health changes, our predictive approach could integrate into a behavioral activation (BA) based intervention. BA is a common treatment, usually for depression, that focuses on finding and increasing behaviors that are positively reinforcing (as opposed to increasing symptoms of depression). For example, the 2018 Intern Health Study had a built-in notification system that tried to promote positive behaviors. Illustrative embodiments herein utilize predicted indicators of resilience to drive targeted interventions that improve resident mental health by increasing resilience early-on within the internship.

Ethics & Privacy

Sensing systems have both positive and negative implications, specifically for residents that have been historically denied employee rights. Since 1999, residents are considered employees under the National Labor Relations Act, and employees have faced heightened workplace discrimination when disclosing mental health status to their employer. Resident physicians often do not seek treatment for mental health because they are specifically worried about the confidentiality of their treatment. Thus, one must be extremely careful when framing how these technologies and the resulting interventions should be integrated into residency programs to protect and support the resident.

There are potential benefits for residents if tracking methodologies are used to build interventions that improve resident resilience. Residency programs have an obligation to provide a positive educational experience to residents, and improved resident well-being leads to improved patient care. For example, any intervention or other remediation action determined or otherwise driven by predicted indicators of resilience as disclosed herein could be codesigned by technologists, residents, and other relevant stakeholders. Within this codesign process, ethical standards should be created by residents to articulate the capabilities and limitations of the technologies deployed, and these standards should become direct requirements of the intervention system. The same approach should be used when designing interventions for employee resilience more broadly.

Using GANs for Wearable Sensing and EMA Prediction

As described above, we developed novel, interpretable MTLCGAN models and applied these models to predict sensor and EMA data. FIG. 6 highlights how using a CGAN and MTL improved model performance for one example feature. The baseline P-MTLREG model in part (a) of FIG. 6 performed well around the mode of the distribution (−0.2≤Cohen's dₛ≤0.0) but could not generate a diverse enough sample space even with participant MTL, a problem that appears equivalent to mode collapse within GANs. Using a CGAN and feature MTL improved the model's ability to generate diverse samples, as the middle column in part (b) of FIG. 6 shows a greater match between the actual and predicted effect size distributions. The right column shows the loss in performance at an individual-level for the F-MTLCGAN, where a low correlation indicated that the model was not able to predict participant-specific feature changes. Part (c) of FIG. 6 shows that the FP-MTLCGAN model improved both sample diversity and individual-level performance.

Overall, the models predicted a lower range of effect sizes than what occurred within the actual data. This may be due to the particular example dataset used for these illustrative embodiments, in which the majority of individuals did not experience large feature changes. Other embodiments can improve the ability to predict individual-level changes by potentially integrating resampling strategies to increase the amount of data within effect size ranges with less participant data.

There were also certain features, specifically the step count, time sleeping, and time in bed, that had much higher correlations with the actual effect sizes compared to the heart rate and mood EMA features. Based upon FIG. 4, a possible explanation for this phenomena is that our example methodology performed better within features whose changes between the BL and INTERN periods were more consistent across individuals. For the mood EMA specifically, it is also possible that the interpolation process could have affected model performance. Nonetheless, it is apparent that GANs as disclosed herein can be applied to predict changes in multivariate probability distributions, given that the predicted and actual changes had significant positive correlations.

An additional point to note is the discrepancy in results between the 1-NN Generated Accuracy and the effect size correlations. The F-MTLCGAN model achieved a lower 1-NN Generated Accuracy than the FP-MTLCGAN model, indicating that the FP-MTLCGAN model was more subject to mode collapse. It is possible that mode collapse did occur within the FP-MTLCGAN model at an individual level, which was not captured by the effect size correlations, but was captured by the 1-NN Generated Accuracy. Alternative embodiments applying generative models to individual-level data streams can be configured to focus on developing metrics that assess model accuracy at both a within and across participant level, if such fine-grained accuracy is needed.

As indicated previously, the modeling approach in illustrative embodiments herein can be applied across a wide variety of sensing datasets. The FP-MTLCGAN model, which resulted in the best performance in some embodiments, utilized Q1 data for pre-cluster matching. It is also possible to utilize this model or a similar model with only BL data, allowing for earlier, more accurate intervention. In addition, adherence to wearing the Fitbit and completing EMA was low per-individual, which prevented us from using models, such as recurrent neural networks, that can also take into account the time varying nature of longitudinal behavioral and physiological features. Alternative embodiments can incorporate methods to raise compliance, so as to allow prediction of more fine-grained changes. Also, interventions and other remediation actions can be codesigned with resident physicians, with an upfront focus on creating ethical and privacy standards for data collection and analysis.

Illustrative embodiments herein provide an accurate and efficient approach to predict indicators of resilience using passive sensing data collected from Fitbit devices and EMA. We formulated novel GANs with an interpretable loss function that applied MTL to predict behavioral, physiological, and well-being changes associated with starting a medical internship. Such embodiments can also determine targeted resilience-building interventions driven by the predicted changes.

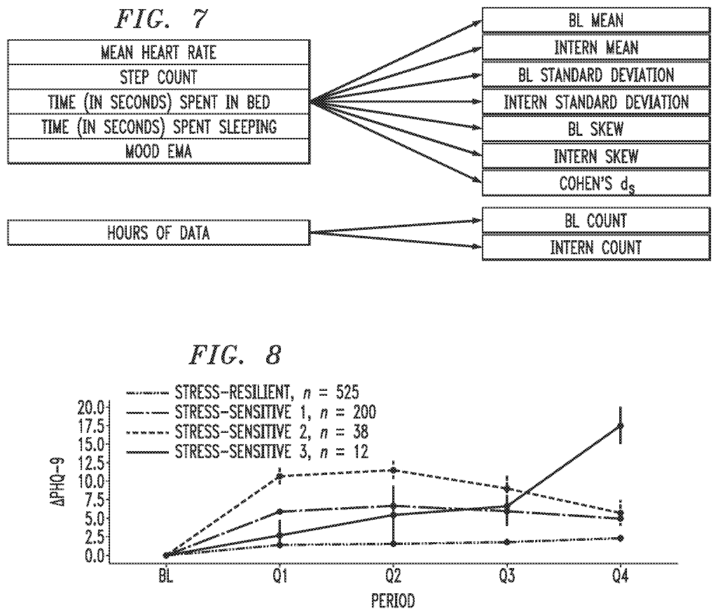

Further details of illustrative embodiments will now be provided with reference to FIGS. 7 through 10. Aspects described below can be applied to one or more of the embodiments previously described, as will be readily apparent to those skilled in the art.

FIG. 7 summarizes indicators used in illustrative embodiments herein. In this example, there are 37 different indicators. Values are either specific to the period before (BL), during (INTERN) the internship, or captured a difference in a specific metric between the INTERN and BL periods (Cohen's d). The indicators on the right are calculated for each metric listed in the same section on the left. For example, we calculated the Mean, Standard Deviation, and Skew for both the BL and INTERN periods, as well as the Cohen's dₛ for the mean heart rate. This results in 7 total indicators for the mean heart rate, and this process can be repeated for each of the 5 hourly features (35 indicators). Two additional indicators were created to capture information about missing data, specifically the count of data per participant in the BL and INTERN periods, resulting in 37 total indicators in this example.

The manner in which these indicators were generated will now be described in more detail. We define the multivariate baseline distribution of hourly features for an individual as A, and the multivariate internship distribution of hourly features for an individual as B. Suppose we have m features, and defining j∈{1, . . . m} Aⱼ and Bⱼ are the distributions for each hourly feature per participant. We computed the mean and standard deviation of the hourly features in both the baseline (X̅_{A}_{ⱼ},SD_{A}_{ⱼ}) and internship X̅_{B}_{ⱼ},SD_{B}_{ⱼ}) distributions. In addition, to create a measure of missing data, we computed the number of hours of total data collected for both the baseline (n_{A}) and internship (n_{B}) periods.

We computed the empirical skew for each feature in both the baseline internship distributions, which is a measure of how “balanced” a distribution is. We expected many of the features in our dataset, such as the mood EMA, to be non-gaussian. We initially created nonparametric indicators (e.g., median and IQR), but found these statistics were highly correlated with their parametric counterparts. Thus, the skew indicator per feature was used to capture how the non-gaussian nature of each feature distribution was associated with stress-resilience. We used the Pearson's skew coefficient, which measures the difference between the empirical mean (X) and the median (v) divided by the standard deviation (SD). For example, for the baseline distribution, a multivariate data point with m features, and a single hourly feature (Aⱼ) for an individual:

$${Skew}_{{A}_{j}} = \frac{3 ( {\overline{X}}_{{A}_{j}} - {v}_{{A}_{j}} )}{{SD}_{{A}_{j}}}$$

Finally, we computed the standardized difference in means, or Cohen's dₛ, between the baseline and internship period, for each hourly feature. We will refer to the vector of Cohen's dₛ for each feature as dₛ, and the Cohen's dₛ for each feature as dₛ_{ⱼ}. For each feature, this value is computed as follows:

$${d}_{{s}_{j}} = \frac{{\overline{X}}_{{A}_{j}} - {\overline{X}}_{{B}_{j}}}{\sqrt{\frac{( {n}_{A} - 1 ) {SD}_{{A}_{j}}^{2} + ( {n}_{B} - 1 ) {SD}_{{B}_{j}}^{2}}{( {n}_{A} + {n}_{B} - 2 )}}}$$

It is to be appreciated that these and other indicators described herein are only examples, and additional or alternative indicators can be used in other embodiments.

Identifying Stress-Resilient and Sensitive Participants

As indicated elsewhere herein, resilience is defined as adaptation to circumstance. We looked to identify a set of individuals within the population whose depression symptoms changed minimally throughout the internship. For example, we can label population subsets that experienced minimal mental health changes as the “stress-resilient” population. By identifying this population, we could then find passive sensing and EMA indicators that distinguished stress-resilient and stress-sensitive individuals. We used quadratic growth mixture models (GMMs) to identify distinct trajectories of depression symptom changes across the population, measured using recorded PHQ-9 changes during baseline and the internship. Such mental health trajectories can be used to distinguish stress-resilient from stress-sensitive individuals. GMMs are similar to linear mixed-effects models, but the key difference is that GMMs identify distinct latent classes within a dataset, and fit a curve to each of these distinct classes. Expectation-maximization is used to optimize both the model parameters and fit classes across individuals as a latent variable. Quadratic models were chosen over linear models, as depression symptoms tend to increase when individuals experienced stress, and decrease after a period of time.

We experimented with identifying 2-5 distinct classes within our example dataset. We then chose the number of classes that minimized both the Akaike Information Criterion (AIC) and the Bayesian Information Criterion (BIC). The resulting AIC and BIC for each pre-defined number of classes can be found in Table 8. We found the 4-class model minimized the AIC (17,127) and BIC (17,215).

TABLE 8
Results from using GMMs to identify different trajectories of
depression symptom changes within our example population.

| # of Classes | AIC | BIC |
|---|---|---|
| 2 | 17,267 | 17,318 |
| 3 | 17,184 | 17,254 |
| 4 | 17,127 | 17,215 |
| 5 | 17,135 | 17,242 |

FIG. 8 shows the depression symptom change (ΔPHQ-9) trajectories from the 4-class quadratic GMM model. Each curve represents the change in depression symptom trajectory for the subset of the population within that class. Points represent the mean ΔPHQ-9 (change in depression symptoms) for that period and population subset represented by that trajectory, and error bars are a 95% confidence interval around the mean. The y-axis, ΔPHQ-9, are the changes in depression symptoms compared to baseline (BL). The x-axis describes the period in which depression symptoms were measured, including the baseline period before the internship (BL), and each quarter, or 3-month period (Q1-4), of the year-long internship. The legend shows the labels for each class, as well as the size of the population subset (n) the trajectories represent. One class was labeled the “stress-resilient” class, because it contained a subset of the population who experienced minimal changes in depression symptoms throughout the internship. More particularly, the majority class (n=525, 68% of participants) who experienced minimal PHQ-9 (depression symptom) changes was qualitatively determined to be the “stress-resilient” population, and the combined other classes (n=250, 32% of participants) were determined to be the “stress-sensitive” population.

Impact of Outliers on Identifying Stress-Resilient Participants

We analyzed if our outlier filtering procedure affected our ability to distinguish stress-resilient versus stress-sensitive individuals. If outlier values were characteristic of stress-sensitivity, we would expect that a higher number of outliers would be filtered out for stress-sensitive compared to stress-resilient participants. A Shapiro-Wilk test showed that the outlier count distribution across participants was non-normally (P<0.05) distributed. A Mann-Whitney U test was performed to examine if the number of outliers identified across stress-sensitive participants was significantly greater than the number of outliers identified across stress-resilient participants. The test was non-significant (U=69, 510.5, P>0.05). We also confirmed that participants were not entirely filtered out of our dataset during the outlier removal procedure. Thus, we believe these outliers did not contain information that distinguished stress-resilient from stress-sensitive individuals.

Identifying Passive Sensing and EMA Indicators of Resilience

Some embodiments herein utilize Generalized Estimating Equations (GEE) to find which of the indicators listed in FIG. 7 significantly differentiated stress-resilient and stress-sensitive individuals.

GEE is a type of linear model that can be applied to measure population effects on clustered or grouped data, and GEE can be more robust compared to other grouped linear models such as linear mixed-effects models because GEE requires less assumptions on the underlying data distributions. Sex and age were controlled for within each model. Sex and age were chosen as controls in some embodiments because these are two characteristics that an individual might be more comfortable to share with an implemented resilience-measurement system, compared to a characteristic like ethnicity, or baseline depression status. We used the internship specialty as the grouping variable, because the intensity of work can vary by specialty. Continuous indicators were standardized by subtracting the mean and dividing by the standard deviation prior to conducting the regression. In addition, a constant term of “1” was added to the regression model as a y-intercept.

GEE parameters, like many linear models, cannot be reliably estimated as a multivariate regression if independent variables are highly correlated. We suspected high correlations within the created 37 potential indicators of resilience of FIG. 7, and thus we conducted our analysis in steps to reduce multicollinearity. We first created separate GEE models for each potential indicator (“univariate GEE”), with controls and specialty groupings. We then filtered to significant (a=0.05) indicators from the univariate GEEs, and began adding each of these indicators to a combined “multivariate GEE” model. Indicators were added by iterating through each hourly feature, and significance. For example, we first would attempt to add the mood EMA most significant indicator, then the seconds in bed most significant indicator, etc. We used this ordering to create more hourly feature diversity within the multivariate GEE model. To reduce multicollinearity, indicators were only added to the multivariate GEE if their variance inflation factor (VIF) between the current indicators within the multivariate GEE was <5. VIF estimates the explained variance for an independent variable from all other independent variables within a model. A VIF>5 means that more than 50% of a variable's variance is explained by all other independent variables currently within a model.

TABLE 9
Results from conducting GEE to determine how each potential passive sensing
and EMA indicator distinguishes stress-resilient and stress-sensitive individuals. As indicated
above, specialty was used as a grouping variable, and we controlled for sex and age in the model.
We list only significant results (α = 0.05) from the univariate regressions. The B_{U} and P_{U} are
the univariate significance and p-value respectively. Multivariate regression was performed after
filtering out highly-correlated indicators. β_{M} and P_{M} are the coefficient and significance values
for the 3 indicators included in the multivariate regression. Values were either specific to the
period before (BL), during (INTERN) the internship, or captured a difference in a specific metric
between the INTERN and BL periods (Difference).

| Hourly | | | | | | |
|---|---|---|---|---|---|---|
| Feature | Metric | Period | β_{U} (95% CI) | P_{U} | β_{M} (95% CI) | P_{M} |
| Step Count | Skew | BL | −0.19 (−0.37 to −0.01) | <.05 | | |
| Step Count | Skew | INTERN | −0.16 (−0.26 to −0.06) | <.01 | −0.16 (−0.26 to −0.06) | <.01 |
| Seconds in | Cohen's dₛ | Difference | 0.15 (0.04 to 0.25) | <.01 | 0.11 (0.00 to 0.22) | <.05 |
| Bed | | | | | | |
| Seconds in | Mean | INTERN | 0.17 (0.08 to 0.27) | <.001 | | |
| Bed | | | | | | |
| Seconds in | Skew | INTERN | 0.15 (0.06 to 0.24) | <.01 | | |
| Bed | | | | | | |
| Seconds in | Standard | INTERN | 0.13 (0.03 to 0.22) | <.05 | | |
| Bed | Deviation | | | | | |
| Heart Rate | Mean | BL | −0.13 (−0.25 to −0.01) | <.05 | | |
| Heart Rate | Mean | INTERN | −0.16 (−0.30 to −0.03) | <.05 | | |
| Seconds of | Cohen's dₛ | Difference | 0.15 (0.05 to 0.25) | <.01 | | |
| Sleep | | | | | | |
| Seconds of | Mean | INTERN | 0.18 (0.09 to 0.27) | <.001 | | |
| Sleep | | | | | | |
| Seconds of | Skew | INTERN | 0.16 (0.07 to 0.25) | <.001 | | |
| Sleep | | | | | | |
| Seconds of | Standard | INTERN | 0.13 (0.03 to 0.23) | <.01 | | |
| Sleep | Deviation | | | | | |
| Mood EMA | Cohen's dₛ | Difference | 0.25 (0.12 to 0.37) | <.001 | 0.26 (0.13 to 0.39) | <.001 |
| Mood EMA | Mean | BL | 0.31 (0.14 to 0.48) | <.001 | | |
| Mood EMA | Mean | INTERN | 0.54 (0.42 to 0.67) | <.001 | | |
| Mood EMA | Standard | BL | −0.23 (−0.33 to −0.12) | <.001 | | |
| | Deviation | | | | | |
| Mood EMA | Standard | INTERN | −0.51 (−0.60 to −0.41) | <.001 | | |
| | Deviation | | | | | |

A positive GEE β coefficient shows a positive association between an indicator and the likelihood an individual is stress-resilient with all other independent variables held constant. The magnitude of the β coefficient can be interpreted as the strength of the association.

Out of the 37 potential indicators, 17 were significantly associated with stress-resilience within the univariate GEEs. For simplicity, we only describe the most significant (P<0.001) indicators below. Having a higher average number of seconds in bed and sleep during the internship increased the likelihood of resilience. Hourly sleep distributions are skewed (most individuals are not sleeping during the day). Increasing the skew translates to the tail of the distribution (more hours with higher seconds of sleep) becoming larger, and this increase in skew during the internship increased the likelihood of stress-resilience. The mood EMA feature showed a number of significant associations with stress-resilience, which was expected given low mood is a direct symptom of depression on the PHQ-9. A higher mood score during the baseline and internship, as well as lower fluctuations in mood (decreased standard deviation) increased the likelihood of stress-resilience. An increased mood score (positive Cohen's dₛ) increased the likelihood of stress-resilience.

After removing correlated features, 3 indicators were included in the multivariate GEE model. The 3 indicators were the INTERN step count skew, seconds in bed Cohen's dₛ, and mood EMA Cohen's dₛ. The high number of filtered indicators showed that the potential indicators were highly correlated. All 3 indicators were significantly associated with resilience, and we describe them further as follows. The step count distributions are skewed because there are many hours during the day when an individual is not moving (hourly step count=0). Thus, decreasing this skew shifts the mode of the distribution away from 0, i.e. there are more hours spent with nonzero step counts. This decrease during the internship period increased the likelihood of stress-resilience (β_{M}=−0.16, P_{M}<0.01). Increasing the amount of time spent in bed increased the likelihood of stress-resilience (β_{M}=0.11, P_{M}<0.05), as well as increasing one's mood (β_{M}=0.26, P_{M}<0.05).

The results in Table 9 show that there were a variety of indicators that summarized both the baseline and internship hourly feature distributions and were significantly associated with distinguishing stress-resilient versus stress-sensitive individuals. An application of this analysis would be to use the found indicators to guide interns towards wellness interventions, or help residency program directors create interventions that improve resilience. Interns may be more willing to engage in these interventions before they are time-constrained by their residency program, and are impacted by internship stress. For example, if the system indicates an individual is less likely to engage in physical activity during the internship, which is linked to higher stress-sensitivity, an intern could begin to build exercise goals into their routine before the internship begins.

In this embodiment, 13 out of 17 of the found indicators were associated with mobile data collected during the internship. These indicators are unknown during the baseline period. We aimed to predict these indicators using mobile data collected during the baseline period, which would be needed for early-assessment and intervention. For some embodiments, we first experimented with regression models, including random forests, gradient boosting trees, and multilayer perceptions, to approximate the resilience indicators from baseline data. We found that these models were unable to achieve accurate predictions across all indicators in some embodiments.

We instead utilized in one or more such embodiments a more complex approach, specifically using density estimation techniques, to predict a multivariate distribution of the hourly features per-individual. Per these multivariate distributions, we would be able to calculate a set of predicted resilience indicators, and verify whether the relationships between the predicted indicators and resilience aligned with the actual indicators and resilience.

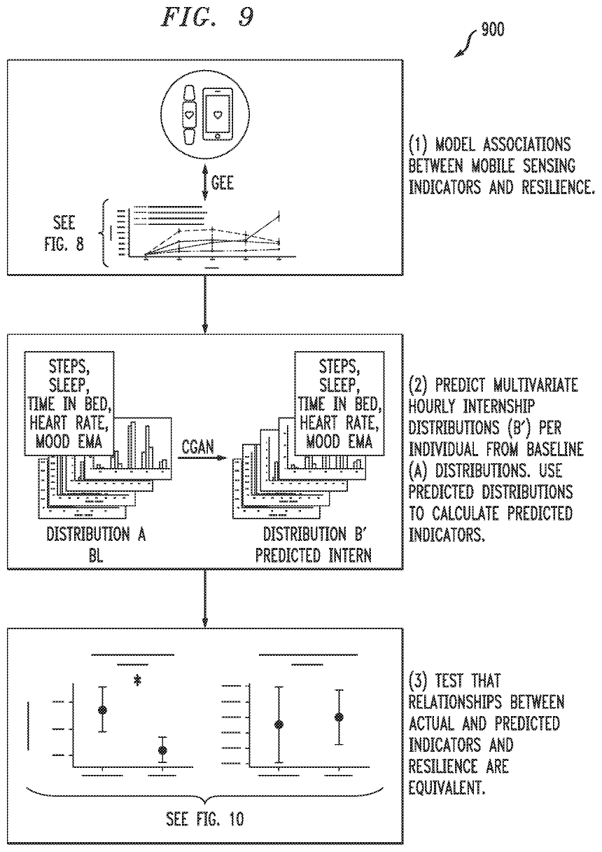

FIG. 9 shows an example analysis pipeline 900 using GEE in an illustrative embodiment. We let A be the multivariate hourly baseline (BL) feature distributions per-individual, and B′ be the predicted multivariate hourly internship (INTERN) feature distribution per-individual. In step (1) we model associations between mobile sensing indicators and resilience using GEE. This involved finding relationships between the actual mobile sensing indicators using both the baseline and internship data and resilience. We then built CGANs in step (2) to predict the internship data (B′) from the baseline data (A) on a per-individual basis. We also calculated predicted mobile sensing indicators using both A and B′. Lastly, in step (3), we validated whether the associations between the predicted indicators and resilience were equivalent to the relationships between the actual indicators and resilience.

Identifying Predicted Passive Sensing and EMA Indicators of Resilience

We performed the univariate GEE analysis described previously with the predicted passive sensing and EMA indicators to explore if the predicted indicators also differentiated stress-resilient and stress-sensitive individuals. The indicators were calculated using internship data generated from the FP-MTLCGAN model, and we used data from both the train and test sets for this analysis. Specifically, we focused on the indicators of resilience identified above. The GEE results are found in Table 9. We did not include any indicators exclusive to the BL period, because they would be equivalent to what was shown in Table 9. Out of the 13 predicted indicators, 5 were significant. This included the seconds in bed skew during the internship, the mean heart rate during-internship, the mood EMA Cohen's dₛ, mean, and standard deviation. The step count and seconds of sleep skew were marginally significant (a=0.10). After conducting the univariate GEE, we conducted the same multivariate GEE described previously, but using the predicted indicators. There were no significant indicators within the multivariate GEE.

TABLE 10
Results from conducting a univariate GEE using each indicator
of resilience identified in Table 9 calculated from the predicted
distributions. The GEE modeled the relationship between predicted
passive sensing and EMA indicators and stress-resilience,
with intern specialty as a grouping variable, and controlling for
age and sex. β_{U} is the coefficient value, and P_{U}
is the significance level. Indicators exclusive to the baseline
(BL) period are not shown because they would have the equivalent
β_{U} coefficient and significance level from Table 9.
Predicted values were either specific to during the internship (INTERN), or captured
a difference in a specific metric between the INTERN and BL periods (Difference).

| Hourly Feature | Metric | Period | β_{U} (95% CI) | | P_{U} |
|---|---|---|---|---|---|
| Step Count | Skew | INTERN | 0.14 | (−0.02 to 0.30) | <.1 |
| Seconds in Bed | Cohen's dₛ | Difference | 0.07 | (−0.05 to 0.19) | |
| Seconds in Bed | Mean | INTERN | 0.10 | (−0.03 to 0.22) | |
| Seconds in Bed | Skew | INTERN | 0.12 | (0.00 to 0.24) | <.05 |
| Seconds in Bed | Standard Deviation | INTERN | 0.04 | (−0.08 to 0.15) | |
| Heart Rate | Mean | INTERN | −0.17 | (−0.29 to −0.05) | <.01 |
| Seconds of Sleep | Cohen's dₛ | Difference | 0.07 | (−0.06 to 0.19) | |
| Seconds of Sleep | Mean | INTERN | 0.09 | (−0.03 to 0.21) | |
| Seconds of Sleep | Skew | INTERN | 0.12 | (−0.00 to 0.24) | <.1 |
| Seconds of Sleep | Standard Deviation | INTERN | 0.04 | (−0.07 to 0.15) | |
| Mood EMA | Cohen's dₛ | Difference | −0.21 | (−0.35 to −0.07) | <.01 |
| Mood EMA | Mean | INTERN | 0.36 | (0.18 to 0.53) | <.001 |
| Mood EMA | Standard Deviation | INTERN | −0.33 | (−0.43 to −0.22) | <.001 |

Comparing Actual and Predicted GEE Coefficients

We then compared the coefficients of the significant predicted (Table 10) and actual (Table 9) passive sensing and EMA indicators associated with differentiating stress-resilient and stress-sensitive individuals. For this comparison, we concatenated the datasets containing the calculated actual and predicted features. We then created two variables: (1) a binary variable that dictated whether a given feature value was from the predicted or actual data, and (2) an interaction term between (1) and the feature values. We then used GEE with the same controls and specialty grouping to explore the associations between these two new features and the original feature for differentiating stress-resilient individuals. The interaction term coefficients modeled the change in the β coefficient when using the actual versus the predicted values for regression. If the coefficient was significant (α=0.05), the difference between the actual and predicted coefficients were significantly different. We conducted this analysis for both the univariate GEE coefficients (β_{U}) and multivariate (β_{M}).

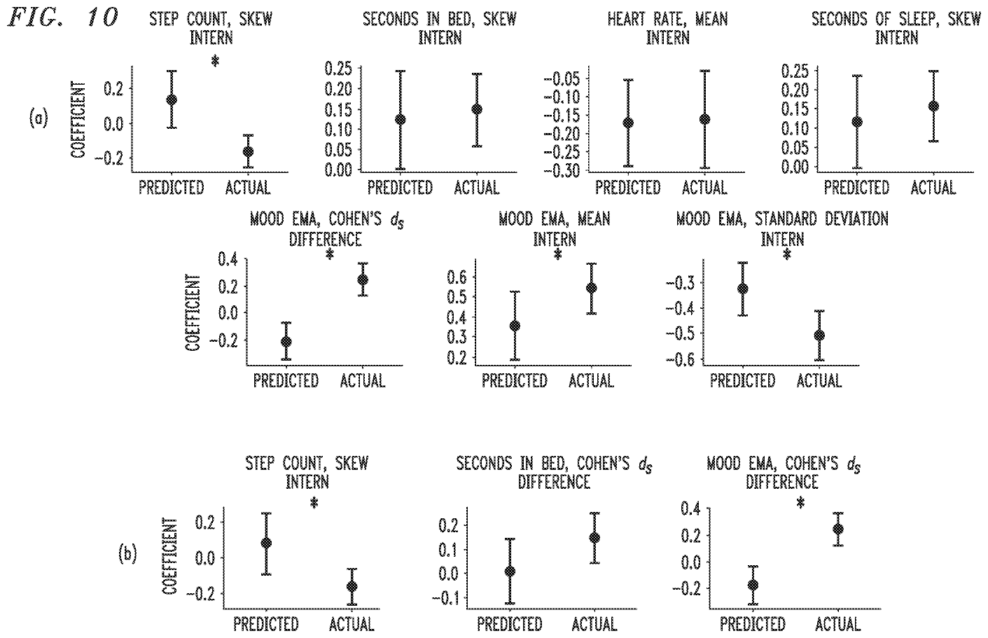

FIG. 10 illustrates the comparison between the actual and predicted coefficients. In part (a) of the figure, the comparison between the actual and predicted coefficients for the univariate GEEs is shown. We found 3 indicator coefficients were not significantly different. These included the seconds in bed skew, the mean heart rate, and the seconds of sleep skew during the internship. Part (b) of the figure shows the comparison between the actual and predicted coefficients for the 3 multivariate GEE indicators. We found 1 feature coefficient that was not significantly different, specifically the seconds in bed Cohen's dₛ.

It should be understood that the particular arrangements shown and described in conjunction with FIGS. 1 through 10 are presented by way of illustrative example only, and numerous alternative embodiments are possible. The various embodiments disclosed herein should therefore not be construed as limiting in any way. Numerous alternative arrangements of GAN-based algorithms can be utilized in other embodiments. Those skilled in the art will also recognize that alternative processing operations and associated system entity configurations can be used in other embodiments.

It is therefore possible that other embodiments may include additional or alternative system elements, relative to the entities of the illustrative embodiments. Accordingly, the particular system configurations and associated algorithm implementations can be varied in other embodiments.

A given processing device or other component of an information processing system as described herein is illustratively configured utilizing a corresponding processing device comprising a processor coupled to a memory. The processor executes software program code stored in the memory in order to control the performance of processing operations and other functionality. The processing device also comprises a network interface that supports communication over one or more networks.

The processor may comprise, for example, a microprocessor, an ASIC, an FPGA, a CPU, a GPU, a TPU, an ALU, a DSP, or other similar processing device component, as well as other types and arrangements of processing circuitry, in any combination. For example, at least a portion of the functionality of at least one GAN or an associated GAN-based prediction and/or remediation algorithm provided by one or more processing devices as disclosed herein can be implemented using such circuitry.

The memory stores software program code for execution by the processor in implementing portions of the functionality of the processing device. A given such memory that stores such program code for execution by a corresponding processor is an example of what is more generally referred to herein as a processor-readable storage medium having program code embodied therein, and may comprise, for example, electronic memory such as SRAM, DRAM or other types of random access memory, ROM, flash memory, magnetic memory, optical memory, or other types of storage devices in any combination.

As mentioned previously, articles of manufacture comprising such processor-readable storage media are considered embodiments of the invention. The term “article of manufacture” as used herein should be understood to exclude transitory, propagating signals. Other types of computer program products comprising processor-readable storage media can be implemented in other embodiments.

In addition, embodiments of the invention may be implemented in the form of integrated circuits comprising processing circuitry configured to implement processing operations associated with implementation of a GAN-based algorithm.

An information processing system as disclosed herein may be implemented using one or more processing platforms, or portions thereof.

For example, one illustrative embodiment of a processing platform that may be used to implement at least a portion of an information processing system comprises cloud infrastructure including virtual machines implemented using a hypervisor that runs on physical infrastructure. Such virtual machines may comprise respective processing devices that communicate with one another over one or more networks.

The cloud infrastructure in such an embodiment may further comprise one or more sets of applications running on respective ones of the virtual machines under the control of the hypervisor. It is also possible to use multiple hypervisors each providing a set of virtual machines using at least one underlying physical machine. Different sets of virtual machines provided by one or more hypervisors may be utilized in configuring multiple instances of various components of the information processing system.

Another illustrative embodiment of a processing platform that may be used to implement at least a portion of an information processing system as disclosed herein comprises a plurality of processing devices which communicate with one another over at least one network. Each processing device of the processing platform is assumed to comprise a processor coupled to a memory. A given such network can illustratively include, for example, a global computer network such as the Internet, a WAN, a LAN, a satellite network, a telephone or cable network, a cellular network such as a 3G, 4G or 5G network, a wireless network implemented using a wireless protocol such as Bluetooth, WiFi or WiMAX, or various portions or combinations of these and other types of communication networks.

Again, these particular processing platforms are presented by way of example only, and an information processing system may include additional or alternative processing platforms, as well as numerous distinct processing platforms in any combination, with each such platform comprising one or more computers, servers, storage devices or other processing devices.

A given processing platform implementing a GAN-based algorithm as disclosed herein can alternatively comprise a single processing device, such as a computer, a mobile telephone, a wearable device, a handheld sensor device, or another type of processing device, that implements not only the GAN-based algorithm but also at least one data source and one or more controlled components. It is also possible in some embodiments that one or more such system elements can run on or be otherwise supported by cloud infrastructure or other types of virtualization infrastructure.

It should therefore be understood that in other embodiments different arrangements of additional or alternative elements may be used. At least a subset of these elements may be collectively implemented on a common processing platform, or each such element may be implemented on a separate processing platform.

Also, numerous other arrangements of computers, servers, storage devices or other components are possible in an information processing system. Such components can communicate with other elements of the information processing system over any type of network or other communication media.

As indicated previously, components of the system as disclosed herein can be implemented at least in part in the form of one or more software programs stored in memory and executed by a processor of a processing device. For example, certain functionality disclosed herein can be implemented at least in part in the form of software.

The particular configurations of information processing systems described herein are exemplary only, and a given such system in other embodiments may include other elements in addition to or in place of those specifically shown, including one or more elements of a type commonly found in a conventional implementation of such a system.

For example, in some embodiments, an information processing system may be configured to utilize the disclosed techniques to provide additional or alternative functionality in other contexts.

It should again be emphasized that the embodiments of the invention as described herein are intended to be illustrative only. Other embodiments of the invention can be implemented utilizing a wide variety of different types and arrangements of information processing systems, networks and processing devices than those utilized in the particular illustrative embodiments described herein, and in numerous alternative processing contexts. In addition, the particular assumptions made herein in the context of describing certain embodiments need not apply in other embodiments. These and numerous other alternative embodiments will be readily apparent to those skilled in the art.

What is claimed is:

1. A method comprising:
obtaining data characterizing a given subject over time;
applying at least a portion of the obtained data to a generative adversarial network adapted to generate a prediction of at least one change in at least one of behavior and physiology of the given subject from the obtained data; and
executing at least one automated remedial action relating to the given subject based at least in part on the generated prediction;
the generative adversarial network being configured to implement multi-task learning, across a plurality of subjects, in which changes in multiple distinct features are treated as separate but linked tasks;
the generative adversarial network comprising separate discriminators for each of the multiple distinct features and separate discriminators for each of a plurality of different clusters of respective subsets of the plurality of subjects;
the generative adversarial network combining outputs of respective ones of the discriminators for the features and outputs of respective ones of the discriminators for the clusters in generating the prediction for the given subject;
wherein the method is performed by at least one processing device comprising a processor coupled to a memory.

2. The method of claim 1 wherein obtaining data characterizing the given subject over time further comprising obtaining data from at least one of:
one or more wearable devices of the given subject;
a smartphone of the given subject; and
one or more sensors associated with the given subject.

3. The method of claim 1 wherein the multiple distinct features comprise one or more of a heart rate measure, a mood measure, a sleep measure and an activity measure, and the generated prediction comprises an indicator of resilience of the given subject under one or more specified conditions, and further wherein the generated prediction is associated with one or more predicted changes in mental health of the given subject so as to permit interpretation of the generated prediction in the context of the mental health of the given subject.

4. The method of claim 1 wherein executing at least one automated remedial action relating to the given subject based at least in part on the generated prediction comprises generating at least one control signal for controlling at least one controlled system component over a network.

5. The method of claim 1 wherein executing at least one automated remedial action relating to the given subject based at least in part on the generated prediction comprises generating at least a portion of at least one output display for presentation on at least one user terminal.

6. The method of claim 1 wherein executing at least one automated remedial action relating to the given subject based at least in part on the generated prediction comprises generating an alert for delivery to at least user terminal over a network.

7. The method of claim 1 wherein executing at least one automated remedial action relating to the given subject based at least in part on the generated prediction comprises generating at least one output signal in a telemedicine application.

8. The method of claim 7 wherein said at least one output signal in a telemedicine application comprises a prediction visualization signal for presentation on a user terminal.

9. The method of claim 7 wherein said at least one output signal in a telemedicine application comprises diagnosis information transmitted over a network to a medical professional.

10. The method of claim 7 wherein said at least one output signal in a telemedicine application comprises prescription information transmitted over a network to a prescription-filling entity.

11. The method of claim 1 wherein the generative adversarial network implements an adversarial loss function that characterizes the generated prediction utilizing a clinically interpretable metric.

12. The method of claim 11 wherein the clinically interpretable metric comprises a Cohen's d metric.

13. The method of claim 1 wherein the clusters of respective subsets of the plurality of subjects are determined by applying a k-means clustering algorithm utilizing a clinically interpretable metric.

14. The method of claim 1 wherein at least a portion of the generative adversarial network is implemented in at least one neural network integrated circuit.

15. A system comprising:
at least one processing device comprising a processor coupled to a memory;
the processing device being configured:
to obtain data characterizing a given subject over time;
to apply at least a portion of the obtained data to a generative adversarial network adapted to generate a prediction of at least one change in at least one of behavior and physiology of the given subject from the obtained data; and
to execute at least one automated remedial action relating to the given subject based at least in part on the generated prediction;
the generative adversarial network being configured to implement multi-task learning, across a plurality of subjects, in which changes in multiple distinct features are treated as separate but linked tasks;
the generative adversarial network comprising separate discriminators for each of the multiple distinct features and separate discriminators for each of a plurality of different clusters of respective subsets of the plurality of subjects;
the generative adversarial network combining outputs of respective ones of the discriminators for the features and outputs of respective ones of the discriminators for the clusters in generating the prediction for the given subject.

16. The system of claim 15 wherein the multiple distinct features comprise one or more of a heart rate measure, a mood measure, a sleep measure and an activity measure, and the generated prediction comprises an indicator of resilience of the given subject under one or more specified conditions, and further wherein the generated prediction is associated with one or more predicted changes in mental health of the given subject so as to permit interpretation of the generated prediction in the context of the mental health of the given subject.

17. The system of claim 15 wherein executing at least one automated remedial action relating to the given subject based at least in part on the generated prediction comprises generating at least one output signal in a telemedicine application.

18. A computer program product comprising a non-transitory processor-readable storage medium having stored therein program code of one or more software programs, wherein the program code, when executed by at least one processing device comprising a processor coupled to a memory, causes the processing device:
to obtain data characterizing a given subject over time;
to apply at least a portion of the obtained data to a generative adversarial network adapted to generate a prediction of at least one change in at least one of behavior and physiology of the given subject from the obtained data; and
to execute at least one automated remedial action relating to the given subject based at least in part on the generated prediction;
the generative adversarial network being configured to implement multi-task learning, across a plurality of subjects, in which changes in multiple distinct features are treated as separate but linked tasks;
the generative adversarial network comprising separate discriminators for each of the multiple distinct features and separate discriminators for each of a plurality of different clusters of respective subsets of the plurality of subjects;
the generative adversarial network combining outputs of respective ones of the discriminators for the features and outputs of respective ones of the discriminators for the clusters in generating the prediction for the given subject.

19. The computer program product of claim 18 wherein the multiple distinct features comprise one or more of a heart rate measure, a mood measure, a sleep measure and an activity measure, and the generated prediction comprises an indicator of resilience of the given subject under one or more specified conditions, and further wherein the generated prediction is associated with one or more predicted changes in mental health of the given subject so as to permit interpretation of the generated prediction in the context of the mental health of the given subject.

20. The computer program product of claim 18 wherein executing at least one automated remedial action relating to the given subject based at least in part on the generated prediction comprises generating at least one output signal in a telemedicine application.
